(19) United States
(12) Patent Application Publication
(10) Pub. No.: US 2013/0156753 A1
(43) Pub. Date: Jun. 20, 2013

(54) THE cAMP/PKA/HDAC5 PATHWAY AND USES THEREOF

(71) Applicant: Zheng-gen Jin, Rochester, NY (US)

(72) Inventor: Zheng-gen Jin, Rochester, NY (US)

(73) Assignee: UNIVERSITY OF ROCHESTER, Rochester, NY (US)

(21) Appl. No.: 13/809,789

(22) PCT Filed: Jul. 31, 2011
(86) PCT No.: PCT/US2011/043873

§ 371 Date: Jan. 11, 2013

Related U.S. Application Data
(60) Provisional application No. 61/363,895, filed on Jul. 13, 2010.

(51) Int. Cl. A61K 39/395; A61K 31/352; A61K 31/7076; A61K 38/10; A61K 31/7088

(52) U.S. Cl. CPC A61K 39/39533; A61K 38/10; A61K 31/7088; A61K 31/7076; A61K 31/352
USPC 424/130.1; 435/6.12; 435/7.1; 435/8; 435/15; 435/29; 435/375; 514/16.4; 514/44 A; 514/47; 514/455

(57) ABSTRACT

The present invention provides novel uses of the cAMP/PKA/HDAC5 pathway for the treatment and prevention of myopathies.

### CROSS REFERENCE TO RELATED APPLICATION

This application claims the benefit of U.S. Patent Application Ser. No. 61/363,895 filed Jul. 13, 2010, the contents of which are incorporated herein by reference in its entirety.

### FEDERALLY SPONSORED RESEARCH OR DEVELOPMENT

The invention was made with government support under grant number HL80611 awarded by the National Institutes of Health. The government has certain rights in the invention.

### BACKGROUND

Myopathic injuries or disorders have profound clinical effects and, in many cases, result in severe disabilities or reduced life spans in patients with the injuries or disorders. Few treatment options are currently available to these patients.

### SUMMARY

Provided herein is a method of inhibiting HDAC5 activity in a cell, comprising contacting the cell with an agent that increases cAMP/PKA/HDAC5 pathway activity in the cell, wherein the cell is subjected to stress. Further provided is a method of preventing cardiac myopathy in a subject, comprising administering to the subject an agent that increases cAMP/PKA/HDAC5 pathway activity. Also provided is a method of preventing or delaying the occurrence of heart failure in a subject, comprising administering to the subject an agent that increases cAMP/PKA/HDAC5 pathway activity.

Further provided is a method of treating heart failure, comprising administering to the subject an agent that decreases cAMP/PKA/HDAC5 pathway activity. Also provided is a method of treating heart failure in a subject comprising administering to the subject an agent that inhibits HDAC5-mediated transcriptional repression of MEF2.

Further provided is a method of identifying a compound that inhibits cardiac myopathy via the cAMP/PKA/HDAC5 pathway comprising contacting a cardiomyocyte with a test compound, wherein the cardiac myocyte is subjected to stress, and measuring phosphorylation of HDAC5 at Ser280. An increase in phosphorylation indicates that the test compound inhibits cardiac myopathy via the cAMP/PKA/HDAC5 pathway. Also provided is a method of identifying a compound that inhibits cardiac myopathy via the cAMP/PKA/HDAC5 pathway. The method comprises contacting a cardiomyocyte with a test compound, wherein the cardiac myocyte is subjected to stress, and measuring the association of HDAC5 with 14-3-3 protein. A decrease in the association between HDAC5 and 14-3-3 indicates that the test compound inhibits cardiac myopathy via the cAMP/PKA/HDAC5 pathway. Further provided is a method of identifying a compound that inhibits the binding of HDAC5 to PKA comprising contacting HDAC5 with a test compound in the presence of PKA and detecting binding of HDAC5 to PKA. A decrease in binding compared to a control indicates that the test compound inhibits binding of HDAC5 to PKA. Also provided is a method of identifying a compound that inhibits binding of HDAC5 to PKA comprising contacting a cell that comprises HDAC5, PKA and a construct containing an MEF2 regulatory region operatively linked to a nucleic acid encoding luciferase with a test compound and detecting the level of luciferase activity. An increase in luciferase activity indicates that the compound inhibits binding of HDAC5 to PKA. Also provided is a method of identifying a compound that inhibits HDAC5-mediated transcriptional repression of MEF2 comprising contacting a cell with a test compound and detecting the level of expression of MEF2-dependent gene(s). An increase in expression of MEF2 dependent genes as compared to control indicates that the test compound is a compound that inhibits HDAC5-mediated transcriptional repression of MEF2.

### DESCRIPTION OF THE DRAWINGS

FIG. 1 shows that PKA inhibits stress signal-regulated HDAC5 nuclear export. A, B, Cos7 cells were co-transfected with expression vectors encoding GFP-tagged HDAC5 (GFP-HDAC5) and constitutively active protein kinases as indicated, and then exposed to phorbol-12-myristate-13-acetate (PMA, 500 μM) for 3 hours. C, D, Cos7 cells were co-transfected with GFP-HDAC5 and then pretreated with the vehicle (DMSO as control), forskolin (10 μM), cAMP (500 μM), cGMP (500 μM), Rolipram (10 μM), IBMX (500 μM), EHNA (30 μM), isoproterenol (1 μM) and cilostamide (5 μM), followed with the exposure of PMA for 3 hours. E, F, Neonatal rat ventricular cardiomyocytes (NRVMs) were infected with an adenoviral expression vector encoding GFP-HDAC5 and then pretreated with the vehicle (DMSO as control), forskolin (10 μM), cAMP (500 μM), cGMP (500 μM), and isoproterenol (1 μM) for 30 min, followed by the exposure of α-adrenergic agonist phenylephrine hydrochloride (PE, 10 μM) for 3 hours. A-F, The cells were fixed and the subcellular localization of GFP-HDAC5 was visualized by fluorescence microscopy. Values represent the percentage of expressing cells in which HDAC5 exhibited nuclear staining Cardiomyocyte protein marker α-actinin immunofluoresence staining and nuclei stained with DAPI were shown. *, p<0.05 versus without PMA or PE, n=4.

FIG. 2 shows that HDAC5 is a novel substrate for PKA. A, Comparison of amino acids surrounding the regulatory serine (arrowhead) between HDAC5 (SEQ ID NO: 2) and HDAC7 (SEQ ID NO: 21) (top), and schematic diagram of the HDAC5 and HDAC7 functional domains (bottom). B, C, Nuclear export of YFP-HDAC7-WT is resistant to forskolin treatment, but nuclear export of YFP-HDAC7-K196S/N197S (mouse sequence) mutant was inhibited by forskolin in Cos7. D, ³²P autoradiograph image from an in vitro kinase assays performed with recombinant PKA-CA and GST-HDAC5-WT (273-286) or GST-HDAC5-S280A (273-286) peptides. The equal loading of GST proteins was shown by Coomassie blue staining E, Cos7 cells were transfected with Flag-tagged HDAC5-WT or Flag-tagged HDAC5-S280A and then treated with forskolin (10 μM) at different time. Phosphorylation of HDAC5 in cell lysates was detected by immunoblotting with PKA phospho-substrate antibodies after immunoprecipitation with anti-Flag antibodies. F, Amino acid sequence alignment of sequence surrounding HDAC5 S280 (SEQ ID NO: 2) in various species. G, Cos7 cells were co-transfected with Flag-HDAC5 and HA-PKA-CA. Coimmunoprecipitation of Flag-HDAC5 antibodies and then immunoblotting with anti-HA, anti-Flag and anti-β-actin antibodies were performed.

FIG. 3 shows that phosphorylation of HDAC5 on Ser280 mediates the inhibition of HDAC5 nuclear export by the cAMP/PKA pathway. A, B, Cos7 cells were transfected with GFP-HDAC5-WT or GFP-HDAC5-S280A, with or without HA-PKA-CA, and then pretreated with cAMP followed with the exposure to PMA for 3 hours. cAMP and PKA-CA inhibited nuclear export of GFP-HDAC5-WT but not GFP-HDAC5-S280A by PMA. C, D, Cos7 cells were transfected with GFP-HDAC5-WT or GFP-HDAC5-S280D and then exposed to PMA for 3 hours. GFP-HDAC5-S280D but not GFP-HDAC5-WT was resistant to PMA-induced nuclear export. *, p<0.05 versus without PMA, n=4. E, F, Cos7 cells were transfected with Flag-HDAC5-WT or Flag-HDAC5-S280A mutant, and then pretreated with forskolin followed with the exposure to PMA. Phosphorylation of HDAC5 in cell lysates was analyzed by immunoblotting with phospho-specific HDAC5 antibodies that recognize the HDAC5 phosphorylated at Ser259 (E) and Ser498 (F). G, H, Cos7 cells were transfected with Flag-HDAC5-WT, Flag-HDAC5-S280A or Flag-HDAC5-S280D, and then pretreated with forskolin, followed with the exposure to PMA. Coimmunoprecipitation of Flag-HDAC5 in cell lysates and then immunoblotting with anti-14-3-3 beta and anti-Flag antibodies in immunoprecipitates were performed. 14-3-3 in total cell lysates (5% of the input) was determined by immunoblotting with anti-14-3-3 antibodies. Representative blots are shown, n=3.

FIG. 4 shows that PKA-dependent HDAC5 phosphorylation and nuclear retention represses MEF2-dependent gene transcription and cardiac fetal gene expression. A, B, C, in Luciferase reporter assays for MEF2 transcriptional activity, NRVMs were infected with adenoviruses expressing 3×MEF2-luciferase reporter gene (A) along with adenoviruses expressing Flag-HDAC5-WT or Flag-HDAC5-S280A, (B), Flag-HDAC5-S280D (C) and then pretreated with forskolin or cAMP for 30 min, followed by stimulation with PE for 24 hours. *, p<0.05 compared with PE+vehicle, n=4. D, E, Semi-quantitative RT-PCR analysis for cardiomyocyte fetal gene expression was performed using NRVMs infected with adenoviruses expressing GFP alone (control), GFP-HDAC5-WT or GFP-HDAC5-S280A for 24 h, and then pretreated with cAMP, followed by stimulation with PE for 24 hours. The mRNA was extracted from the cell lysates and then RT-PCR with the primers for ANF, β-MHC, α-SMA, and GADPH (internal control) was performed. *, p<0.05 versus PE+Ad-GFP; #, p<0.05 versus PE+GFP-HDAC5-WT, n=4.

FIG. 5 shows that PKA-dependent HDAC5 phosphorylation and nuclear retention inhibits cardiomyocyte hypertrophy. A-H, Cardiomyocyte size was detected by immunostaining with anti-α-actinin antibody. NRVMs were pretreated with cAMP for 30 min and then treated with PE for 24 hours (A, B, C). NRVMs were infected with adenoviruses expressing GFP-HDAC5-WT, GFP-HDAC5-S280D (D, E, F), or GFP-HDAC5-S280A (G, H), and then pretreated with the vehicle (DMSO as control), cAMP for 30 min, followed by the exposure of PE for 24 hours. The cells were fixed and analyzed for GFP-HDAC5 localization, α-actinin staining and nuclear DAPI staining *, p<0.05 versus without PE; #, p<0.05 versus with PE alone. I, Schematic model for PKA-dependent regulation of HDAC5 subcellular localization and gene transcription.

FIG. 6 shows that PKA inhibits PKD/CaMK-induced HDAC5 nuclear export in COS7 cells. Cos7 cells were co-transfected with expression vectors encoding GFP-tagged HDAC5 and the constitutively active form of PKD1, CaMK-I, and/or HA-PKA-CA.

FIG. 7 shows that PKI inhibits forskolin/cAMP effects on PMA-induced HDAC5 Nuclear Export in Cos7 cells. Cos7 cells were transfected with plasmids GFP-tagged HDAC5 and then pretreated with the vehicle (DMSO as control) and PKA inhibitor 14-22 amide (PKI 14-22 Amide) and then treated with forskolin or cAMP, followed by the exposure of PMA for 3 hours. The cells were fixed and GFP-HDAC5 localization was analyzed by fluorescence microscopy.

FIG. 8 shows that PKA inhibits stress signal-regulated HDAC5 nuclear export in adult rat ventricular cardiomyocytes (ARVMs). ARVMs were pretreated with vehicle (DMSO as control) and cAMP (500 μM) for 30 min, followed by exposure of a adrenergic agonist phenylephrine hydrochloride (PE, 10 μM) for 3 hours. The cells were fixed and endogenous HDAC5 localization was analyzed by fluorescence microscopy. HDAC5 was detected by immunofluorescence using an antibody against HDAC5 (Santa Cruz sc-11419) (Santa Cruz Biotechnology, Santa Cruz, Calif.) and a fluorescein-conjugated secondary antibody.

FIG. 9 shows that PKI inhibits forskolin/cAMP effects on PE-induced HDAC5 Nuclear Export in NRVMs. NRVMs were infected with an adenoviral expression vector encoding GFP-tagged HDAC5 and then pretreated with the vehicle (DMSO as control) and PKI 14-22 Amide, and then treated with forskolin or cAMP, followed by exposure with phenylephrine hydrochloride (PE) for 3 hours. The cells were fixed and GFP-HDAC5 localization was analyzed by fluorescence microscopy. α-actinin was detected by immunofluorescence using an antibody against α-actinin and a fluorescein-conjugated secondary antibody. The nuclei were stained with DAPI.

FIG. 10 shows that PKACA siRNA inhibits cAMP effects on PE-induced HDAC5 Nuclear Export in NRVMs. A, NRVMs were transfected with PKACA siRNA and then infected with adenoviral expression vector encoding GFP-tagged HDAC5, and then pretreated with the vehicle (DMSO as control) and cAMP, followed by exposure with phenylephrine hydrochloride (PE) for 3 hours. The cells were fixed and GFP-HDAC5 localization was analyzed by fluorescence microscopy. α-actinin was detected by immunofluorescence using an antibody against α-actinin and a fluorescein-conjugated secondary antibody. The nuclei were stained with DAPI. B, PKA expression in cell lysates were determined by Western blot using PKA antibody.

FIG. 11 shows that nuclear localized PKA inhibits HDAC5 nuclear export in COS7 cells. CoS7 cells were cotransfected with expression vectors encoding GFP-tagged HDAC5 and HcRed1-NUC, or HcRed1-NUCPKA-CA in the absence or presence of PMA.

FIG. 12 shows that PKA inhibits endogenous HDAC5 nuclear export in NRVMs. NRVMs were pretreated with vehicle (DMSO as control) and cAMP (500 μM) for 30 min, followed by the exposure of α-adrenergic agonist phenylephrine hydrochloride (PE, 10 μM) for 3 hours. The cells were fixed and endogenous HDAC5 localization was analyzed by fluorescence microscopy. HDAC5 and α-actinin was detected by immunocytochemistry with antibody against HDAC5 and α-actinin.

FIG. 13 shows the alignment of human HDAC5 (SEQ ID NO: 19) and human HDAC7 (SEQ ID NO: 20). There is about 50% identity between the amino acid sequence of HDAC5 and the amino acid sequence of HDAC7. The “RRSS” motif in HDAC5 is replaced by “RRKN” in HDAC7.

FIG. 14 shows that PKACA phosphorylates HDAC5 at Serine 280. ³²P autoradiograph image from in vitro kinase assays performed with recombinant PKA-CA and Myc-HDAC5 AA 1-360. Myc-HDAC5 AA 1-360 were transfected into Cos7 cells, and then immunoprecipitated with Myc antibody. The equal loading of Myc-proteins was shown by Ponceau S staining

FIG. 15 shows that nuclear export of HDAC5-S280A mutant is resistant to PKA inhibition in cardiomyocytes. NRVMs were infected with an adenoviral expression vector encoding GFP-tagged HDAC5 S280A and then pretreated with vehicle (DMSO as control), cAMP (500 μM), and forskolin (10 μM) for 30 min, followed by the exposure of PE for 3 hours. The cells were fixed and GFP-HDAC5 subcellular localization was analyzed by fluorescence microscopy and stained with an α-actinin antibody and DAPI.

FIG. 16 shows that the HDAC5-S280D mutant mimics a PKA effect on HDAC5 nuclear export in cardiomyocytes. NRVMs were infected with an adenoviral expression vector encoding GFP-tagged HDAC5 WT or GFP-tagged HDAC5 S280D, and then treated with PE for 3 hours. The cells were fixed and GFP-HDAC5 localization was analyzed by fluorescence microscopy and stained with an alpha-actinin antibody and DAPI.

FIG. 17 shows that Forskolin (PKA activator) did not affect PKD1-dependent HDAC5 phosphorylation at Ser259 and Ser498 sites. Cos7 cells were co-transfected with plasmids Flag-HDAC5-WT or Flag-HDAC5-S280A and constitutively active HA-PKD1, and then treated with forskolin. Immunoblots of HDAC5 phosphorylation at Ser259 and Ser498 sites were shown.

FIG. 18 shows that overexpression of HDAC5 dose-dependently decreases the level of endogenous HDAC5. NRVMs were infected with an adenoviral expression vector encoding GFP-tagged HDAC5 WT or S280A at different doses. The endogenous HDAC5 and exogenous GFP-HDAC5s were analyzed by immunoblotting.

FIG. 19 shows that cAMP decreases PE-induced ANF expression in cardiomyocytes. NRVMs were pretreated with cAMP for 30 min, followed by stimulating with PE for additional 24 hours. The mRNA was extracted from the cell lysates, and quantitative RT-PCR with the primers for ANF and GADPH (internal control) were performed as described in the Examples. Quantitative data are shown (n=4).

FIG. 20 shows that overexpression of HDAC5-S280D inhibits cardiomyocyte hypertrophy in NRVMs. Cardiomyocyte size was detected by immunostaining with anti-α-actinin antibody. NRVMs were treated with PE for 24 hours (A, B, C). NRVMs were infected with adenoviruses expressing GFP-HDAC5-WT, GFPHDAC5-S280D and then treated with the vehicle (DMSO as control) and PE for 24 hours. The cells were fixed and analyzed for GFP-HDAC5 localization, α-actinin staining

FIG. 21 shows that PKA activators inhibit PE/Angiotensin II-induced cardiomyocyte hypertrophy. NRVMs were pretreated with the vehicle (DMSO as control), PKA activator forskolin (10 μM), cAMP (500 μM), and Rolipram (10 μM) for 30 min, followed by the exposure of PE or Angiotensin II for 24 hours. Cardiomyocyte protein marker α-actinin was detected by immunofluorescence using antibody against α-actinin and fluorescein-conjugated secondary antibody.

FIG. 22 is a schematic model for PKA inhibition on HDAC5 nucleocytoplasmic shuttling and gene expression. A, HDAC5 represses transcriptional expression of target genes by binding with MEF2. B, Activated PKD or CaMK phosphorylates HDAC5 at serine 259 and serine 498 resulting in export of HDAC5 to the cytoplasm as a complex with 14-3-3. The displacement of HDAC5 from MEF2 allows for transcriptional expression of the MEF2-dependent genes. C, Activated PKA translocates to the nucleus, and then phosphorylates HDAC5 at serine 280 and CREB at serine 133. D, Phosphorylation of HDAC5 by PKA blocks PKD/CaMK-dependent HDAC5 association with 14-3-3 resulting in HDAC5 nuclear retention and consequent repression of MEF2-dependent genes.

FIG. 23 shows that HDAC5 does not inhibit CREB transcriptional activity in COS7 cells. NRVMs were co-transfected with an expression vector encoding CREB-luciferase reporter gene along with expression vector encoding Flag-HDAC5-WT or Flag-HDAC5-S280D for 48 h and then treated with forskolin. Error bars indicate standard errors (n=3).

### DETAILED DESCRIPTION

Described herein are methods and compositions related to a cAMP/PKA/HDAC5 pathway previously unrecognized to be involved in cellular myopathy. cAMP, cyclic AMP or 3′-5′-cyclic adenosine monophosphate is a second messenger important in many biological processes. cAMP activates protein kinase A (PKA, cAMP-dependent protein kinase), which phosphorylates substrate proteins, such as, for example, HDAC5, a histone deacetylase.

Provided herein is a method of inhibiting HDAC5 activity in a cell, comprising contacting the cell with an agent that increases cAMP/PKA/HDAC5 pathway activity in the cell, wherein the cell is subjected to stress. Thus, the method provides for increasing cAMP/PKA/HDAC5 pathway activity at any of several points. For example, by increasing cAMP activity, by increasing PKA activity, by increasing phosphorylation of HDAC5 or any combination thereof.

A cell used in the methods taught herein can be any cell that comprises at least the components of the cAMP/PKA/HDAC5 pathway, i.e. cAMP, PKA and HDAC5. For example, the cell can be a myocyte, such as a cardiomyocyte. Optionally, the cell is recombinantly modified to include one or more of these components. The cell can also comprise cellular proteins that interact with cAMP, PKA or HDAC5. For example, the cell can comprise proteins that bind to cAMP, PKA or HDAC5 or act upstream or downstream of cAMP, PKA or HDAC5. The cell can be any eukaryotic cell, for example, from a mammal. The cell can be part of an organism, or part of a cell culture, such as a culture of mammalian cells. The cell can also be part of a population or subpopulation of cells. The cell can be in vitro, ex vivo or in vivo. Therefore, the cell can be in a subject.

In the methods set forth herein, cellular stress can be caused by oxidation, physical stress, a chemotherapeutic, a drug, a hormone, pro-inflammatory cytokines, immunostimulatory molecules, heat, ethanol, genetic polymorphisms or mutations that result in cellular stress and the like. Cellular stress can occur before, during or after contacting the cell with the agent.

As set forth throughout, HDAC5 activity can be any activity of HDAC5, for example, nuclear export, the interaction between HDAC5 and a 14-3-3 protein, an activity associated with myocyte enhancer factor-2(MEF2) dependent gene expression, or an activity associated with cardiac fetal gene expression, to name a few. Therefore, inhibition of an HDAC5 activity can be, for example, inhibition of nuclear export or inhibition of the interaction between HDAC5 and a 14-3-3 protein. It is noted that increased activation of the cAMP/PKA/HDAC5 pathway leads to repression of MEF2-dependent gene expression and inhibition of cardiac fetal gene expression, thus preventing cellular myopathy. Methods of measuring HDAC5 activity are set forth in the Examples. These are merely exemplary as numerous methods of measuring protein-protein interactions (for example, Western blot, immunoprecipitation and immunofluorescence) and gene expression (for example, quantitative PCR, RT-PCR, microarray and the like) are available to those of skill in the art.

As utilized throughout, inhibition or a decrease does not have to be complete as this can range from a slight decrease to complete ablation of an HDAC5 activity. For example, a decrease can be at least about 10%, 20%, 30%, 40%, 50%, 60, 70%, 80%, 90%, 95%, 100% or any other percentage decrease in between these percentages as compared to a control. An increase can be any percentage up to and in excess of 100%. For example, in any of the methods described herein, HDAC5 activity can be compared to HDAC5 activity in a control cell not contacted with the compound. HDAC5 activity can also be compared to HDAC5 activity in the same cell prior to addition of the agent or after the effect of the agent has subsided.

As utilized throughout, an agent can be a chemical, a small or large molecule (organic or inorganic), a drug, a protein, a peptide, a cDNA, an antibody, an aptamer, a morpholino, a triple helix molecule, an siRNA, a shRNA, an miRNA, an antisense RNA, a ribozyme or any other compound now known or identified in the future that increases cAMP/PKA/HDAC5 pathway activity. The agent can also be also a combination of two or more of the compositions described herein.

For example, the agent can be a PKA activator, such as forskolin or cAMP. The agent can also be an agent that increases cAMP levels, for example, a cAMP dependent PDE IV inhibitor, a PDE II inhibitor, an adenylate cyclase activator, a Gₛ activator or a beta-adrenergic agonist. Examples of cAMP dependent PDE IV inhibitors include, but are not limited to rolipram, 3-isobutyl-1-methylxanthine (IBMX), mesembrine, ibudilast, luteolin, drotaverine and aminophylline. Examples of PDE II inhibitors include erythro-9-(2-hydroxy-3-nonyl)adenine, BAY 60-7550, PDP (9-(6-Phenyl-2-oxohex-3-yl)-2-(3,4-dimethoxybenzyl)-purin-6-one) and Oxindole. Examples of adenylate cyclase activators include, but are not limited to, forskolin and PACAP 27 amide. Examples of beta-adrenergic agonists include, but are not limited to, Dobutamine, Isoproterenol, Xamoterol, epinephrine, albuterol, Fenoterol, Formoterol, Metaproterenol, Salmeterol, Terbutaline, Clenbuterol, Isoetarine, pirbuterol, procaterol and ritodrine.

### Treatment Methods

Also provided herein is a method of preventing a myopathy in a subject, comprising administering to the subject an agent that increases cAMP/PKA/HDAC5 pathway activity. A myopathy is a disease or condition which causes a decrease in muscle function. Myopathies can be inherited, inflammatory, or caused by endocrine problems. Skeletal muscle myopathy and cardiomyopathy are examples of myopathies that can be prevented by increasing cAMP/PKA/HDAC5 pathway activity. Cardiomyopathy is a decrease in the function of the myocardium for any reason. Examples of cardiomyopathies include, but are not limited to, dilated cardiomyopathy, hypertrophic cardiomyopathy, arrhythmogenic right ventricular cardiomyopathy, noncompaction cardiomyopathy, ischemic cardiomyopathy and valvular cardiomyopathy. Causes of cardiomyopathy include congenital heart disease, nutritional diseases, hypertension, inflammation, cardiomyopathy secondary to a systemic metabolic disease, alcohol, diabetes, cardiomyopathy secondary to cancer, and cardiomyopathy secondary to viral, bacterial or parasitic infection, to name a few. It is understood that by preventing cardiomyopathy in a subject, subsequent events, for example, heart failure, a heart attack, a stroke or cardiac arrest, that can be precipitated by cardiomyopathy can also be prevented. Thus, also provided herein is a method of preventing or delaying the occurrence of heart failure in a subject, comprising administering to the subject an agent that increases cAMP/PKA/HDAC5 pathway activity.

As utilized herein, by prevent,preventing, or prevention is meant a method of precluding, delaying, averting, obviating, forestalling, stopping, or hindering the onset, incidence, severity, or recurrence of cardiomyopathy. For example, the disclosed method is considered to be a prevention if there is a reduction or delay in onset, incidence, severity, or recurrence of cardiomyopathy, or one or more symptoms of cardiomyopathy (e.g., shortness of breath, dizziness, lightheadedness, swelling in the ankles, feet, or legs, fatigue, chest pain, heart murmur, to name a few) in a subject susceptible to cardiomyopathy compared to control subjects susceptible to cardiomyopathy that did not receive an agent that increases cAMP/PKA/HDAC5 pathway activity. The disclosed method is also considered to be a prevention if there is a reduction or delay in onset, incidence, severity, or recurrence of cardiomyopathy, or one or more symptoms of cardiomyopathy (e.g., shortness of breath, dizziness, lightheadedness, swelling in the ankles, feet, or legs, fatigue, chest pain, heart murmur, to name a few) in a subject susceptible to cardiomyopathy after receiving an agent that increases cAMP/PKA/HDAC5 pathway activity as compared to the subject's progression prior to receiving treatment. Thus, the reduction or delay in onset, incidence, severity, or recurrence of cardiomyopathy can be about a 10, 20, 30, 40, 50, 60, 70, 80, 90, 100%, or any amount of reduction in between.

Further provided herein is a method of preventing a neurodegenerative disease or diabetes in a subject, comprising administering to the subject an agent that increases cAMP/PKA/HDAC5 pathway activity. Further provided herein is a method of preventing type II diabetes in a subject, comprising administering to the subject an agent that increases cAMP/PKA/HDAC5 pathway activity. Neurodegenerative diseases include, but are not limited to Alzheimer's disease, Parkinson's disease, Huntington's disease and Amyotrophic lateral sclerosis.

As used throughout, by subject is meant an individual. Preferably, the subject is a mammal such as a primate, and, more preferably, a human. Non-human primates include marmosets, monkeys, chimpanzees, gorillas, orangutans, and gibbons, to name a few. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Veterinary uses and formulations for same are also contemplated herein.

Further provided is a method of treating heart failure in a subject, comprising administering to the subject an agent that decreases cAMP/PKA/HDAC5 pathway activity. Once a subject exhibits one or more symptoms of heart failure or has been diagnosed with heart failure, inhibition of cAMP/PKA/HDAC5 pathway activity can be effected, for example, by decreasing cAMP levels, decreasing binding of PKA to HDAC5, by decreasing HDAC5-mediated transcriptional repression of MEF2 or by decreasing phosphorylation of HDAC5. Also provided herein is a method of treating heart failure in a subject comprising administering to the subject an agent that inhibits binding of HDAC5 to PKA. Further provided is a method of treating heart failure in a subject comprising administering to the subject an agent that inhibits phosphorylation of HDAC5 at Ser280 by PKA. Also provided is a method of treating heart failure in a subject, comprising administering to the subject an agent that inhibits HDAC5-mediated transcriptional repression of MEF2.

Throughout this application, by treat,treating, or treatment is meant a method of reducing the effects or delaying progression of an existing condition, for example, heart failure. Treatment can also refer to a method of reducing the disease or condition itself rather than just the symptoms. The treatment can be any reduction from native levels and can be, but is not limited to, the complete ablation of the condition or the symptoms of the condition. Treatment can range from a positive change in a symptom or symptoms of heart failure to complete amelioration of the condition as detected by art-known techniques. For example, a disclosed method is considered to be a treatment if there is about a 10% reduction in one or more symptoms of the disease in a subject with the disease when compared to native levels in the same subject or control subjects. Thus, the reduction can be about a 10, 20, 30, 40, 50, 60, 70, 80, 90, 100%, or any amount of reduction in between as compared to native or control levels.

The methods can also result in a decrease in the amount of time that it normally takes to see improvement in a subject. For example, this can be a decrease of hours, a day, two days, three days, four days, five days, six days, seven days, eight days, nine days, ten days, eleven days, twelve days, thirteen days, fourteen days, fifteen days or any time in between that it takes to see improvement in the symptoms, or any other parameter utilized to measure improvement in a subject. For example, if it normally takes 7 days to see improvement in a subject not taking the composition, and, after administration of the composition, improvement is seen at 6 days, the composition is an effective treatment. This example is not meant to be limiting as one of skill in the art would know that the time for improvement will vary depending on the condition.

Also provided herein is a method of treating heart failure by diagnosing with heart failure a subject that is taking an agent that increases cAMP/PKA/HDAC5 pathway activity, and substituting the agent that increases cAMP/PKA/HDAC5 pathway activity with an agent that decreases cAMP/PKA/HDAC5 pathway activity in order to treat heart failure.

An agent can be a chemical, a small or large molecule (organic or inorganic), a drug, a protein, a peptide, a cDNA, an antibody, an aptamer, a morpholino, a triple helix molecule, an siRNA, a shRNA, an miRNA, an antisense RNA, a ribozyme or any other compound now known or identified in the future that decreases cAMP/PKA/HDAC5 pathway activity. In particular, an agent that inhibits HDAC5 activity, for example, HDAC5 binding to PKA, or HDAC5-mediated transcriptional repression of MEF2 is provided herein for the treatment of heart failure. For example, anti-HDAC5 antibodies as well as antisense, siRNA, or miRNA directed against HDAC5 are contemplated herein for the treatment of heart failure. The agent can also be also a combination of two or more of the compositions described herein. Agents that decrease cAMP levels can be administered. These include NSAIDs such as ibuprofen, aspirin, acetaminophen as well as beta adrenergic antagonists, for example, acebutolol, carteolol, celiprolol, mepindolol, oxprenolol, pindolol, carvedilol, nebivolol and propanolol. PKA inhibitors such as TTYADFIASGRTGRRNAIHD (SEQ ID NO: 1), Rp-cAMPS and H89 can also be administered to decrease cAMP/PKA/HDAC5 pathway activity.

In another example, an antibody that selectively binds the PKA binding site of HDAC5 can be administered. In particular, antagonistic anti-HDAC5 antibodies and antagonistic anti-PKA antibodies are contemplated herein. In another example, an antibody that selectively binds the HDAC5 binding site on PKA can be administered. The antibody can be a polyclonal antibody or a monoclonal antibody. The antibody can also be humanized. The antibody optionally selectively binds a polypeptide. By selectively binds or specifically binds is meant an antibody binding reaction, which is determinative of the presence of the antigen. Thus, under designated immunoassay conditions, the specified antibodies bind preferentially to a particular peptide and do not bind in a significant amount to other proteins in the sample. Preferably, selective binding includes binding at about or above 1.5 times assay background and the absence of significant binding is less than 1.5 times assay background. In particular, antibodies that specifically bind KVAERRSSPLLRRK (SEQ ID NO: 2) or a fragment thereof are contemplated herein.

Peptides that inhibit or antagonize cAMP/PKA/HDAC5 pathway activity can also be administered. These peptides can be any peptide in a purified or non-purified form, such as peptides made of D-and/or L-configuration amino acids. Peptides that can be utilized include but are not limited to KVAERRSSPLLRRK (SEQ ID NO: 2) or a fragment thereof. Fragments comprising at least five, six, seven, eight, nine or ten consecutive amino acids of SEQ ID NO: 2 are also provided as well as peptides comprising an amino acid sequence having at least 80% identity with SEQ ID NO: 2 or a fragment thereof. Also provided are peptides comprising an amino acid sequence having at least 80%, 85%, 90%, 95%, or 99% identity with SEQ ID NO: 2 or a fragment thereof. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted using the local algorithm of Smith and Waterman Adv. Appl. Math. 2: 482 (1981), by the alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/bl2seq/bl2.html) or by inspection.

The peptides can also be part of a fusion protein comprising another peptide or protein. For example, a peptide set forth herein can be fused to a targeting moiety such as a nuclear localization signal to effect nuclear targeting of the peptide.

The agents described herein can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include buffers such as phosphate buffers, citrate buffer, and buffers with other organic acids;

antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Administration can be carried out using therapeutically effective amounts of the agents described herein for periods of time effective to treat or prevent myopathic disorders or injuries.

The effective amount may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

Any appropriate route of administration may be employed, for example, parenteral, intravenous, subcutaneous, intramuscular, intracranial, intraorbital, convection enhanced delivery, ophthalmic, intraventricular, intracapsular, intraspinal, intracisternal, intraperitoneal,vaginal, rectal, intranasal, aerosol, or oral administration. Administration can be systemic or local. Pharmaceutical compositions can be delivered locally to the area in need of treatment, for example by topical application or local injection. Multiple administrations and/or dosages can also be used. Effective doses can be extrapolated from dose-response curves derived from in vitro or animal model test systems.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

In an example in which a nucleic acid is employed, such as an antisense or an siRNA molecule, the nucleic acid can be delivered intracellularly (for example by expression from a nucleic acid vector or by receptor-mediated mechanisms), or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). siRNA carriers also include, polyethylene glycol (PEG), PEG-liposomes, branched carriers composed of histidine and lysine (HK polymers), chitosan-thiamine pyrophosphate carriers, surfactants (for example, Survanta and Infasurf), nanochitosan carriers, and D5W solution. The present disclosure includes all forms of nucleic acid delivery, including synthetic oligos, naked DNA, plasmid and viral delivery, whether integrated into the genome or not.

Physical transduction techniques can also be used, such as liposome delivery and receptor-mediated and other endocytosis mechanisms (see, for example, Schwartzenberger et al., Blood 87:472-478, 1996) to name a few examples. This methods can be used in conjunction with any of these or other commonly used gene transfer methods.

### Screening Methods

Provided herein is a method of identifying a compound that inhibits cardiac myopathy via the cAMP/PKA/HDAC5 pathway comprising contacting a cardiomyocyte with a test compound, wherein the cardiac myocyte is subjected to stress, and measuring phosphorylation of HDAC5 at Ser280. An increase in phosphorylation indicates that the test compound inhibits cardiac myopathy via the cAMP/PKA/HDAC5 pathway.

This method can further comprise measuring the size of the cardiomyocyte, wherein a decrease in size indicates that the test compound inhibits cardiac myopathy. Examples of methods for measuring the size of cardiomyocytes are set forth in the Examples below.

This method optionally further comprises measuring nuclear export of HDAC5, wherein a decrease in nuclear export indicates that the test compound inhibits cardiac myopathy.

Further provided herein is a method of identifying a compound that inhibits cardiac myopathy via the cAMP/PKA/HDAC5 pathway comprising contacting a cardiomyocyte with a test compound, wherein the cardiac myocyte is subjected to stress, and measuring the association of HDAC5 with 14-3-3 protein. A decrease in the association between HDAC5 and 14-3-3 indicates that the test compound inhibits cardiac myopathy via the cAMP/PKA/HDAC5 pathway. This method can further comprise measuring the size of the cardiomyocyte, wherein a decrease in size indicates that the test compound inhibits cardiac myopathy. This method can also further comprise measuring nuclear export of HDAC5, wherein a decrease in nuclear export indicates that the test compound inhibits cardiac myopathy.

Any cardiac myocyte comprising at least cAMP, PKA and HDAC5 can be used, including cells that have been recombinantly modified to express one or more of the components of the cAMP/PKA/HDAC5 pathway. Populations of cardiac myocytes can also be used. Cellular stress can occur by any means. For example the cell can be contacted with a chemotherapeutic, a drug, a hormone, a pro-inflammatory cytokine, an immunostimulatory molecule, heat or ethanol. Physical stress can also be applied. These examples are not meant to be limiting as one of the skill in the art would know that a variety of methods are available for effecting cellular stress. The cell can be contacted with the compound before cellular stress, during cellular stress, i.e., simultaneously, or after cellular stress.

In the methods set forth herein, phosphorylation can be compared to phosphorylation levels in a control cell not contacted with the test compound. Phosphorylation levels can also be compared to phosphorylation levels in the same cell prior to addition of the test compound or after the effect of the compound has subsided.

Similarly, if the size of the cardiomyocyte(s) is measured, the size of the cardiomyocyte(s) can be compared to the size of a control cell(s) not contacted with the test compound. The size of the cardiomyocyte(s) can also be compared to the size of the same cardiomyocyte(s) prior to addition of the test compound or after the effect of the compound has subsided.

Furthermore, if nuclear export is measured, nuclear export of HDAC5 can be compared to nuclear export of HDAC5 in a control cell not contacted with the test compound. Nuclear export of HDAC5 can also be compared to nuclear export of HDAC5 in the same cell prior to addition of the test compound or after the effect of the compound has subsided.

Also, if the association of HDAC5 with 14-3-3 protein is measured, HDAC5/14-3-3 association can be compared to HDAC5/14-3-3 association in a control cell not contacted with the test compound. HDAC5/14-3-3 association can also be compared HDAC5/14-3-3 association in the same cell prior to addition of the test compound or after the effect of the compound has subsided.

Also provided is a method of identifying a compound that inhibits the binding of HDAC5 to PKA comprising contacting HDAC5 with a test compound in the presence of PKA and detecting binding of HDAC5 to PKA, a reduction in binding compared to a control indicating that the test compound inhibits binding of HDAC5 to PKA. The binding assay can be a cellular assay or a non-cellular assay in which HDAC5, PKA and the compound are brought into contact, for example, via immobilization of HDAC5 and PKA on a column, and subsequently contacting the immobilized complex with the compound, or vice versa. Standard yeast two hybrid screens are also suitable for identifying a compound that inhibits a protein-protein interaction.

Also provided is a method of identifying a compound that inhibits binding of HDAC5 to PKA comprising contacting a cell that comprises HDAC5, PKA and a construct containing an MEF2 regulatory region operatively linked to a nucleic acid encoding luciferase with a test compound. Luciferase activity is detected, an increase in luciferase activity indicating that the compound inhibits binding of HDAC5 to PKA.

Further provided herein is a method of identifying a compound that inhibits HDAC5-mediated transcriptional repression of MEF2 comprising contacting a cell with a test compound and detecting the level of expression of MEF2-dependent genes, wherein an increase in expression of MEF2 dependent genes as compared to control indicates that the test compound is a compound that inhibits HDAC5-mediated transcriptional repression of MEF2. This method can further comprise measuring HDAC5 activity or phosphorylation of HDAC5 and correlating the level of HDAC5 activity and/or phosphorylation of HDAC5 with expression of MEF2-dependent genes. The MEF2 genes that can be measured include, but are not limited to, ANF and cardiac α-actin. The assay can be performed via microarray. Microarray Techniques for constructing arrays and methods of using these arrays are described in EP No. 0 799 897; PCT No. WO 97/29212; PCT No. WO 97/27317; EP No. 0 785 280; PCT No. WO 97/02357; U.S. Pat. Nos. 5,593,839; 5,578,832; EP No. 0 728 520; U.S. Pat. No. 5,599,695; EP No. 0 721 016; U.S. Pat. No. 5,556,752; PCT No. WO 95/22058; and U.S. Pat. No. 5,631,734. Commercially available polynucleotide arrays, such as Affymetrix GeneChip™, can also be used. Use of the GeneChip™ to detect gene expression is described, for example, in Lockhart et al., Nature Biotechnology 14:1675 (1996); Chee et al., Science 274:610 (1996); Hacia et al., Nature Genetics 14:441, 1996; and Kozal et al., Nature Medicine 2:753, 1996.

Any of the compounds identified by the methods set forth herein can be utilized to treat or prevent a myopathic disorder or injury (including a cardiac disorder or injury), neurodegenerative disease or diabetes in a subject. Thus, provided herein are methods of making therapeutic agents using compounds identified by these methods and methods of preventing or treating subjects using the identified compounds or therapeutic agents.

As used in the specification and the appended claims, the singular forms a, an and the include plural referents unless the context clearly dictates otherwise. The term or refers to a single element of stated alternative elements or a combination of two or more elements, unless the context clearly indicates otherwise. As used herein, comprises means includes. Thus, comprising A or B, means including A, B, or A and B, without excluding additional elements.

The following examples are put forth so as to provide those of ordinary skill in the art with a complete disclosure and description of how the compounds and/or methods claimed herein are made and evaluated, and are intended to be purely exemplary of the invention and are not intended to limit the scope of what the inventors regard as their invention except as and to the extent that they are included in the accompanying claims. Efforts have been made to ensure accuracy with respect to numbers (e.g., amounts, temperature, etc.), but some errors and deviations should be accounted for.

### EXAMPLES

### Cell Culture

Cos7 cells were grown in Dulbecco's Modified Eagle's Medium (DMEM) supplemented with fetal bovine serum (10%), 1-glutamine and penicillin/streptomycin. Primary cultures of neonatal rat cardiac ventricular myocytes (NRVMs) from 1- to 2-day-old Sprague-Dawley rats (Charles River Laboratories) were prepared. In brief, NRVMs were dispersed from the ventricles by digestion with collagenase type II (Worthington). Cells were cultured in Dulbecco's modified Eagle's medium (DMEM) supplemented with 1,000 mg/L D-glucose, L-glutamine, and 110 mg/L sodium pyruvate (GIBCO), fetal bovine serum (10%), and penicillin/streptomycin at 37° C. in a 5% CO2 humidified atmosphere for 2-3 days. For inducer or inhibitor studies, cells were pretreated with various inducers or inhibitors for 30 min in serum-free medium. Primary cultures of adult rat cardiomyocytes were prepared from 200-250g male Sprague-Dawley rats. Briefly, rats were anesthetized, hearts excised and placed in Krebs buffer and perfused and digested with endotoxin-free collagenase II (Worthington). This protocol typically yielded approximately 4×10⁶ cells per heart, with ˜85% surviving, rod-shaped Ca2+-tolerant cells. Myocytes were allowed to attach to 20 μg/ml laminin coated plates in Modified Eagle Medium (MEM) containing 2.5% FBS, 25 mmol/L HEPES, 5 mmol/L taurine, 2 mmol/L carnitine, 2 mmol/L creatinine, 100 IU/ml penicillin, and 100 μg/ml streptomycin for 2 hrs before replacing with serum-free MEM containing 25 mmol/L HEPES, 5 mmol/L taurine, 2 mmol/L carnitine, 2 mmol/L creatinine, 100 IU/ml penicillin, and 100 μg/ml streptomycin. Myocytes were pretreated with cAMP for 30 min and stimulated with PE for 3 hrs.

### Materials

PMA (phorbol-12-myrisstate-13-acetate), Forskolin, Dibutyryl-cAMP, DibutyrylcGMP, Rolipram, Isoproterenol, Cilostamide, IBMX, EHNA and PKI (PKA Inhibitor 14-22 Amide) were from Calbiochem (San Diego, Calif.). PE (R-(−)-Phenylephrine) was from Sigma. The antibodies for phospho-PKA substrate were purchased from Cell Signaling Technologies. The antibodies for 14-3-3 β (c-20) and HA-probe (Y-11) were from Santa Cruz, Inc. The antibodies for α-Actinin (sarcomeric), DAPI and Flag-probe were from Sigma. The antibody for phospho-HDAC5 Ser259 was kindly provided by Dr. Timothy McKinsey, and the antibody for phospho-HDAC5 Ser498 was purchased from Signalway AntiBody (SAB 11193-1). HDAC5 antibody was from Santa Cruz (sc-11419).

### Plasmids and Transient Transfection

GFP-HDAC5-WT, FLAG-HDAC5-WT, and YFP-HDAC7-WT have been described previously (Ha C H, et al. (2008) Protein Kinase D-dependent Phosphorylation and Nuclear Export of Histone Deacetylase 5 Mediates Vascular Endothelial Growth Factor-induced Gene Expression and Angiogenesis. J Biol Chem 283(21):14590-14599; Ha C H et al. (2008). VEGF stimulates HDAC7 phosphorylation and cytoplasmic accumulation modulating matrix metalloproteinase expression and angiogenesis. Arterioscler Thromb Vasc Biol 28(10):1782-1788).

HDAC4, HDAC9, and constitutively active mutants of PKA, PKG, MEK1, Akt, MKK3, MKK4, IKK-β, and CAMK1 were purchased from Addgene Inc (Canbridge, Mass.). GFP-HDAC4 and GFP-HDAC9 GFPHDAC5-S280A and GFP-HDAC5-S280D were generated from GFP-HDAC5-WT with the Quick-change site-directed mutagenesis kit (Stratagene (Agilent, Santa Clara, Calif.)) and confirmed by DNA sequencing. For the S280A mutant, oligonucleotides 5′-GCTGAGCGGAGAAGCGCTCCCCTCCTGCGT-3′ (SEQ ID NO:3) and 5′-ACGCAGGAGGGGAGCGCTTCTCCGCTCAGC-3′ (SEQ ID NO:4) were used as primers for PCR. For S280D, the primers were 5′-GCTGAGCGGAGAAGCGATCCCCTCCTGCGT-3′ (SEQ ID NO:5) and 5′-ACGCAGGAGGGGATCGCTTCTCCGCTCAGC-3′(SEQ ID NO:6). By identical methods, YFP-HDAC7-K279S/N280S was generated from YFP-HDAC7-WT. For YFPHDAC7-K279S/N280S,the primers were 5′-CCTGGAGAGACGCAGCAGTCCCCTGCTCAGGA-3′ (SEQ ID NO:7) and 5′-TCCTGAGCAGGGGACTGCTGCGTCTCTCCAGG-3′(SEQ ID NO:8). Flag-HDAC5-S280A and Flag-HDAC5-S280D were generated from GFP-HDAC5-S280A and GFP-HDAC5-S280D, respectively. HA-PKA-CA-NLS was generated from HA-PKA-CA by PCR cloning. For HA-PKA-CA-NLS, primers were 5′-GCTAGTCCGGAATGTACCCATACGATG TTCCAGATTACGCTATGGGCAACGCCGCCGCCGCCAA-3′ (SEQ ID NO:9) and 5′-GCCCTCGAGCAAACTCAGTAAACTCCTTGCCA-3′ (SEQ ID NO:10). HcRed1-PKA-CANLS was generated from HA-PKA-CA-NLS. HDAC5 peptides containing amino acids 273-286 of WT and S280A mutant were cloned into pGEX5X-2 vector for in vitro phosphorylation assay. Transient transfection of plasmid DNA was performed using lipofectamine 2000 (Invitrogene, Carlsbad, Calif.) and 0.5 μg of the indicated plasmids.

### Adenoviral Construction and Infection

Adenoviruses encoding Flag-tagged HDAC5 wild-type (WT), GFP-tagged HDAC5-WT, and YFP-tagged HDAC7-WT were generated as described previously (Ha C H, et al. (2008) Protein Kinase D-dependent Phosphorylation and Nuclear Export of Histone Deacetylase 5 Mediates Vascular Endothelial Growth Factor-induced Gene Expression and Angiogenesis. J Biol Chem 283(21):14590-14599; Ha C H et al. (2008) VEGF stimulates HDAC7 phosphorylation and cytoplasmic accumulation modulating matrix metalloproteinase expression and angiogenesis. Arterioscler Thromb Vasc Biol 28(10):1782-1788). Adenovirus expressing GFP-tagged HDAC5-S280A mutant, GFP-tagged HDAC5-S280D mutant and Flag-tagged HDAC5-S280A mutant were generated by using ViraPower Adenoviral Expression System (Invitrogen) according to the manufacturer's protocol. Adenovirus containing β-galactosidase (LacZ) or GFP was used as a control. NRVMs and Cos7 cells were infected with adenovirus expressing indicated proteins at the indicated multiplicity of infection (MOI) for 24 hours, and then treated with or without inhibitors or inducers for 30 min followed by the application of PMA or PE.

Small Interference RNA (siRNA) and its Transfection

The siRNA duplex targeting rat PKACA and the scrambled siRNA control (a non-targeting siRNA pool) were purchased from Dharmacon, Inc (Chicago, Ill.). For transfection of siRNA, NRVMs were transfected with PKACA siRNA using lipofectamine 2000 (Invitrogen) according to the manufacturer's protocol PE stimulation was performed 48 hour after siRNA transfection.

### Fluorescence Images (Subcellular Localization)

For analysis of GFP-HDAC5 subcellular localization in NRVMs and Cos7 cells, cells were plated in the presence of adenovirus (multiplicity of infection, 50 to 100) on 35 mm dishes containing DMEM plus 10% FBS as previously described (Vega R B, et al. (2004) Protein kinases C and D mediate agonist-dependent cardiac hypertrophy through nuclear export of histone deacetylase 5. Mol Cell Biol 24(19):8374-8385).

After 24 hour culture, cells were maintained in serum-free DMEM medium for additional 24 hours, and then exposed to various inducers or inhibitors (for 30 minutes) prior to PE or PMA stimulation for indicated times. The cells were washed with PBS and fixed with 3.7% formaldehyde in PBS. Images were captured at a magnification of ×60, using a fluorescence microscope (Olympus BX51) equipped with a digital camera and Spot software system (RT Color Diagnostic Instruments). For analysis of the myocyte marker, sarcomeric α-actinin antibody and DAPI in neonatal cardiomyocyte, immunocytochemistry were performed according to a standard protocol. For subcellular localization of endogenous HDAC5 in cardiomyocyes, we fixed cells and used the detergent Saponin (Sigma, 84510) to make cells permeable, and then performed immunocytochemistry with anti-HDAC5 antibody (Santa Cruz, sc-11419). For subcellular localization of HDAC5, total around 200 cells were counted in each condition. For cardiomyocyte size, total myocyte surface areas were calculated using NIH Image J software, and expressed as the average of 100 randomly selected cells per condition.

### In Vitro Phosphorylation Assay

For in vitro PKA-induced phosphorylation assays, GST-fusion peptide of HDAC5-WT or GST-tagged peptide of HDAC5-S280A were mixed in 500 μl of phosphate buffered saline (PBS) and incubated for 30 min at 4° C. Slurry of glutathione-Sepharose 4B was subsequently added, followed by further incubation for 1 hour at 4° C. GST-fusion peptides were incubated 20 min at 30° C. in a 50 μl final volume that contained 50 mM Tris-HCl pH 7.5, 10 mM MgCl2, 200 μM ATP, 0.1 mM {γ-³²P}ATP (5 μCi/tube) and 100 ng of purified catalytic subunit of protein kinase A (PKA-CA, Calbiochem, NJ, USA). Reactions were terminated by addition of SDS sample buffer followed by boiling. The eluates were resolved by SDS-PAGE, transferred to PVDF membranes and visualized by autoradiography.

### Western Blot and Immunoprecipitation

Cells were harvested in lysis buffer and clarified by centrifugation as described previously (Ha C H, et al. (2008) Protein Kinase D-dependent Phosphorylation and Nuclear Export of Histone Deacetylase 5 Mediates Vascular Endothelial Growth Factor-induced Gene Expression and Angiogenesis. J Biol Chem 283(21)). The protein concentrations in the lysates were determined using the Bradford method (BioRad, Hercules, Calif.). Immunoprecipitation were performed according to standard protocols as described previously (Ha C H, et al. (2008) Protein Kinase D-dependent Phosphorylation and Nuclear Export of Histone Deacetylase 5 Mediates Vascular Endothelial Growth Factor-induced Gene Expression and Angiogenesis. J Biol Chem 283(21)). The immune complex samples or total cell lysates were resolved on SDS-PAGE according to standard protocols (Ha C H, et al. (2008) Protein Kinase D-dependent Phosphorylation and Nuclear Export of Histone Deacetylase 5 Mediates Vascular Endothelial Growth Factor-induced Gene Expression and Angiogenesis. J Biol Chem 283(21)). For Western blots, the protein samples from total cell lysates were separated by SDS-PAGE, transferred to nitrocellulose membrane and incubated with appropriate primary antibodies. After incubating with fluorescence-conjugated secondary antibodies, immunoreactive proteins were visualized by Odyssey Infrared Imaging System (LI-COR Biotechnology, Nebraska). Densitometric analyses of immunoblots were performed with Odyssey software (LICOR Biotechnology). Results were normalized by arbitrarily setting the densitometry of control sample to 1.0.

### Luciferase Assay

To assess MEF2 and CREB transcriptional activation, an adenovirus was used that encoded a 3×MEF2-dependent reporter gene in which three tandem repeats of MEF2 sites were located upstream of the thymidine kinase gene promoter and plasmid encoding CREB-dependent reporter gene. NRVMs or Cos7 cells cultured in 24-well were co-transfected with 3×MEF2 or CREB luciferase reporter gene and adenovirus encoding LacZ or GFP-HDAC5-WT, GFP-HDAC5-S280A mutant and GFP-HDAC5-S280D mutant. At 48 hours post-transfection, cells were treated with forskolin or cAMP for 30 min followed by PE for another 24 hours. The luciferase activities in cell lysates were determined using the Dual-Luciferase Reporter Assay kit (Promega) and Wallac 1420 multi-label counter (PerkinElmer) as described previously (4, 5).

### RT-PCR

Total RNA was isolated from NRVM using using TRIzol (Invitrogen Corp.). Firststrand cDNA was synthesized with the SuperScript Preamplification System (Gibco-BRL). cDNA was amplified by PCR for 30 cycle (Applied Biosystem) as described previously (4, 5). Rat GAPDH served as an internal control. The following oligonucleotide primers were used in this study: rat ANF, sense 5′-ATGGGCTCCTTCTCCATCAC-3′ (SEQ ID NO:11) and antisense 5′-ATCTTCGGTACCGGAAGCTG-3′(SEQ ID NO:12) rat α-SMA, sense 5′-ACTGGGACGACATGGAAAAG-3′ (SEQ ID NO:13) and antisense 5′-CATCTCCAGAGTCCAGCACA-3′ (SEQ ID NO:14); rat β-MHC (myosin heavy chain), sense 5′-CCTCGCAATATCAAGGGAAA-3′ (SEQ ID NO:15) and antisense 5′-TACAGGTGCATCAGCTCCAG-3′ (SEQ ID NO:16); rat GAPDH, sense 5′-CGATGCTGGCGCTGAGTA-3′ (SEQ ID NO:17) and antisense 5′-CGTTCAGCTCAGGGATGACC-3′ (SEQ ID NO:18). Experiments were repeated three times.

### Statistics Analysis

All values in the text and figures are expressed as means±SEM at least three independent experiments from given n sizes. The significance of the results was assessed by a paired t-test between two groups. Differences among 3 or more groups were analyzed by contrast analysis, using the Super ANOVA. A p value<0.05 was considered significant.

### Results

These studies show that cyclic adenosine 3′, 5′-monophosphate (cAMP)-activated protein kinase A (PKA) specifically phosphorylates HDAC5 and prevents its nuclear export leading to suppression of gene transcription. PKA directly interacts with HDAC5 and phosphorylates HDAC5 at serine 280, an evolutionarily conserved site. Phosphorylation of HDAC5 by PKA interrupts the association of HDAC5 with protein chaperone 14-3-3 and hence inhibits stress signal-induced nuclear export of HDAC5. An HDAC5 mutant that mimics PKA-dependent phosphorylation localizes in the nucleus and acts as a dominant inhibitor for myocyte enhancer factor 2 transcriptional activity. Molecular manipulations of HDAC5 show that PKA-phosphorylated HDAC5 inhibits cardiac fetal gene expression and cardiomyocyte hypertrophy. The findings disclosed herein identify HDAC5 as a novel substrate of PKA, and reveal a cAMP/PKA-dependent pathway that controls HDAC5 nucleocytoplasmic shuttling and represses gene transcription. This pathway represents a new mechanism by which cAMP/PKA signaling modulates a wide range of biological functions and human diseases such as cardiomyopathy.

The cyclic adenosine 3′, 5′-monophosphate (cAMP)/protein kinase A (PKA) signaling pathway regulates a variety of cellular functions and numerous important biological processes. In these studies, it was found that cAMP/PKA signaling represses gene transcription and cardiomyocyte hypertrophy by phosphorylating HDAC5 and presenting its nuclear export.

### PKA Prevents HDAC5 Nuclear Export

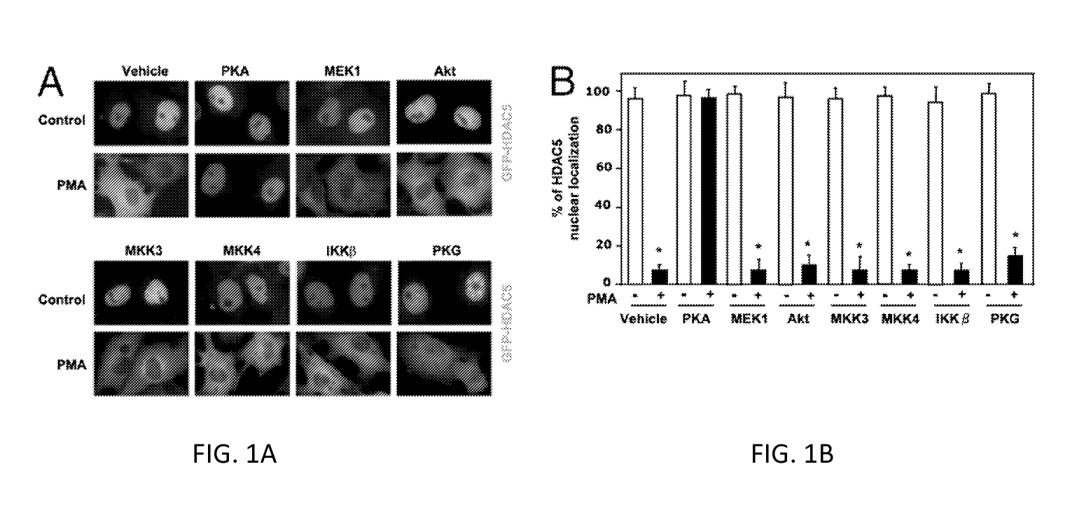

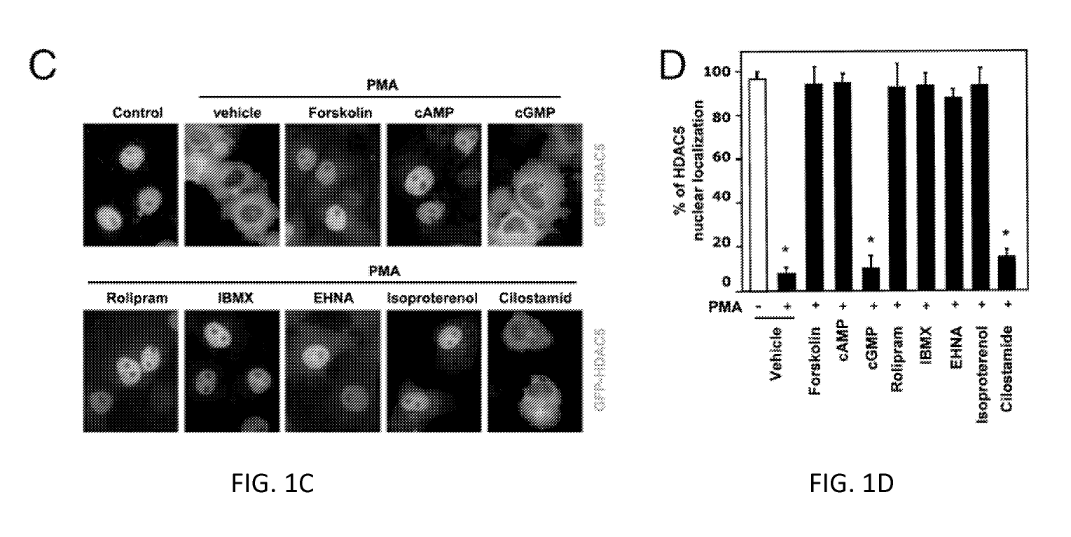

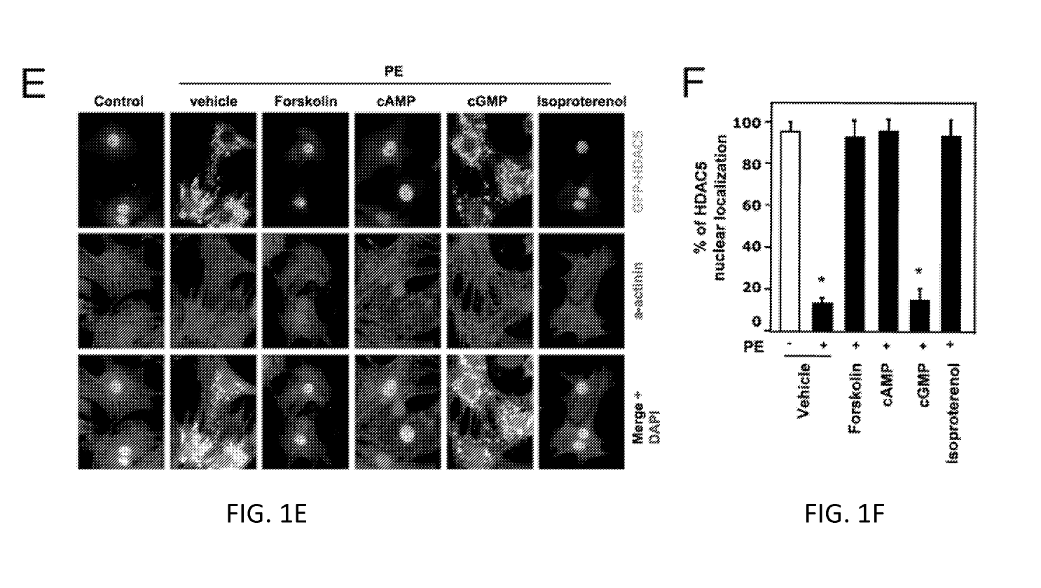

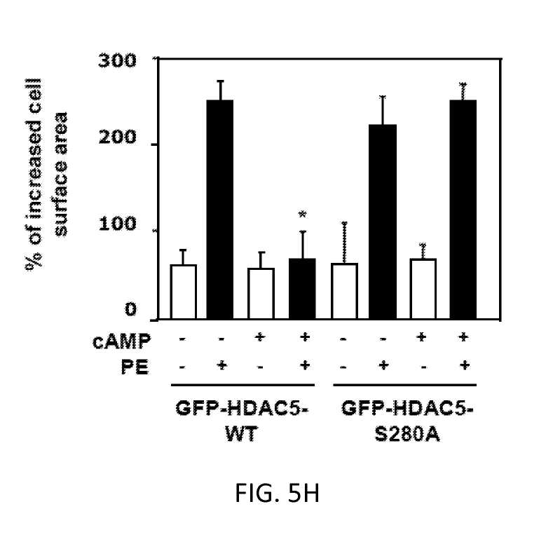

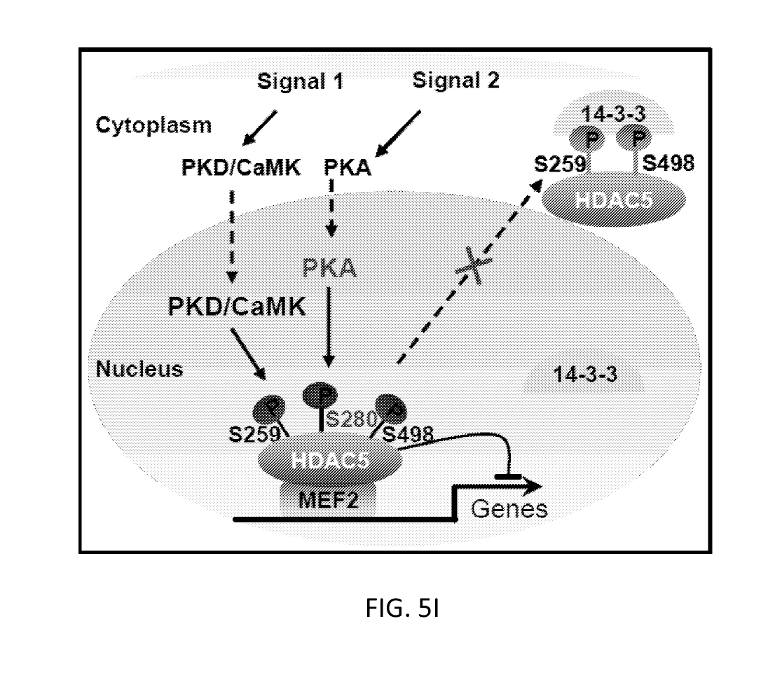

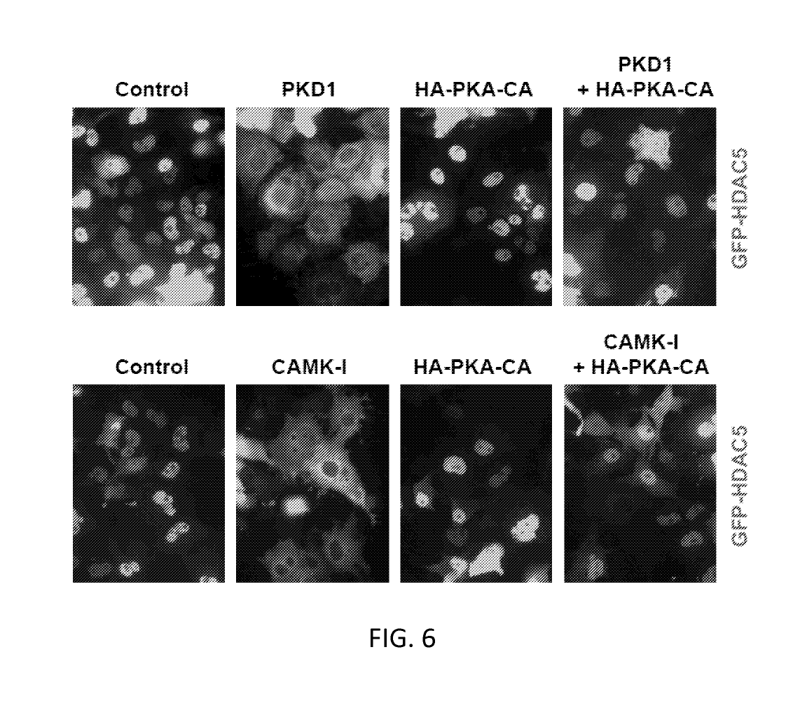

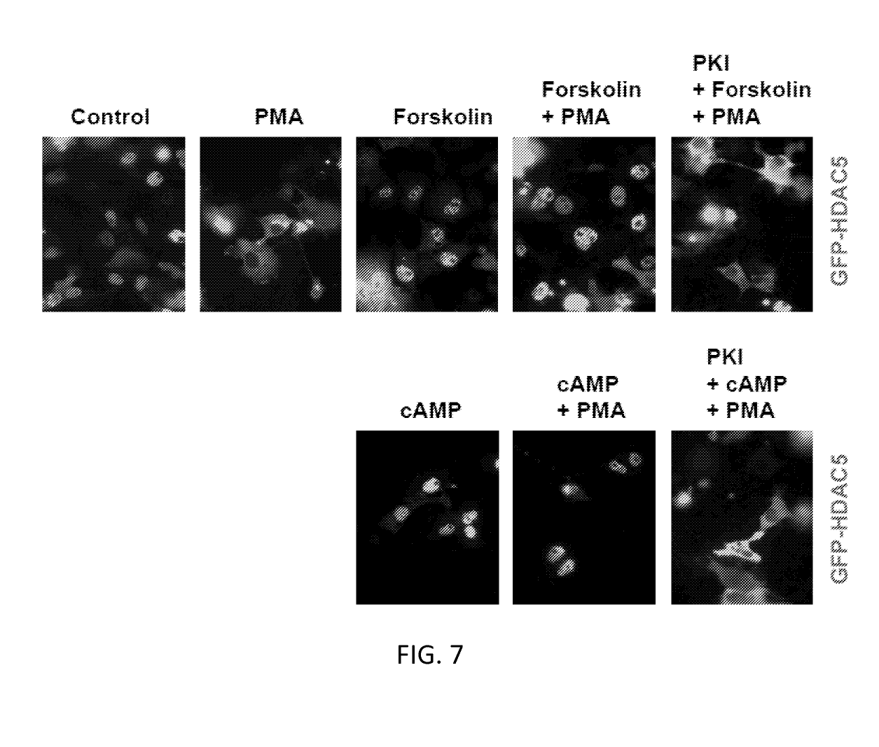

To search for possible protein kinases inhibiting HDAC5 nuclear export, the effects of various protein kinases on HDAC5 nucleocytoplasmic shuttling were examined. Cos7 cells were co-transfected with GFP-tagged HDAC5 and various constitutively active kinases followed with the treatment with phorbol 12-myristate 13-acetate (PMA), a well-documented stimulus for nuclear export of class IIa HDAC. Interestingly, it was found that PKA catalytic subunit (PKA-CA) blocked PMA-induced nuclear export of HDAC5 (FIGS. 1A and B). Other kinases tested in these studies did not have an inhibitory effect on nucleocytoplasmic shuttling of HDAC5. Furthermore, co-transfection experiments show that PKA-CA also inhibited PKD- and CaMK-induced HDAC5 nuclear export in Cos7 cells (FIG. 6). Consistent with the PKA effect, the PKA activators forskolin and cAMP totally blocked HDAC5 nuclear export (FIGS. 1C and D). The effects of forskolin and cAMP are PKA-dependent because a specific PKA inhibitor PKI (PKA inhibitor 14-22 Amide) abolished the inhibition of forskolin and cAMP on HDAC5 nuclear export (FIG. 7). The treatment of phosphodiesterase (PDE) inhibitors rolipram (cAMP-specific PDE IV Inhibitor), IBMX, EHNA (PDE II Inhibitor) and β-adrenergic receptor (β-AR) agonist isoproterenol that increase intracellular cAMP level, also inhibited HDAC5 nuclear export (FIGS. 1C and D). However, cGMP and selective cGMP-specific PDE inhibitor cilostamide did not block HDAC5 nuclear export, indicating the specificity of the cAMP/PKA pathway for regulation of HDAC5 nuclear export.

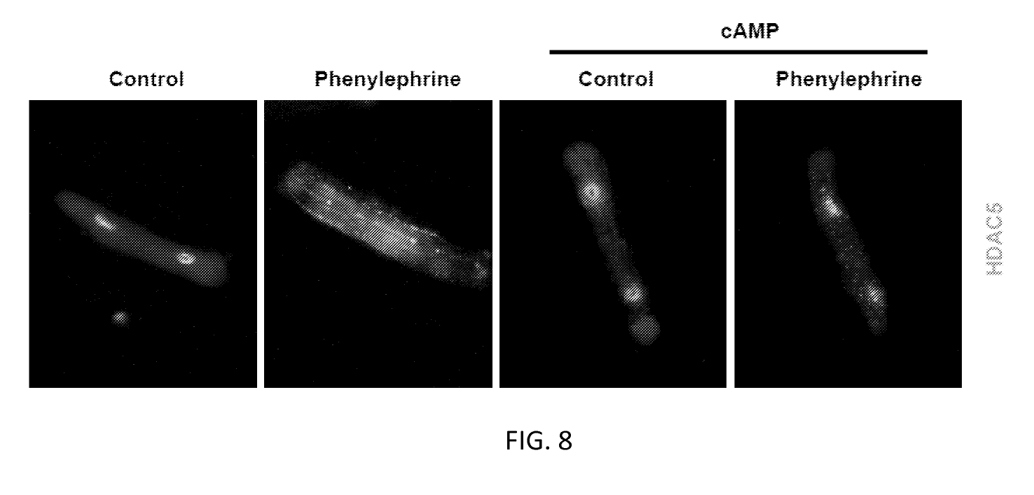

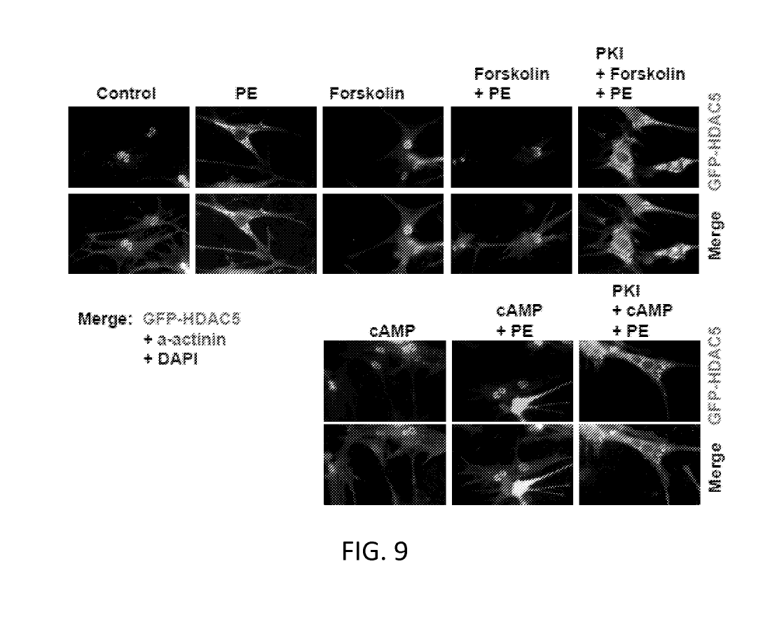

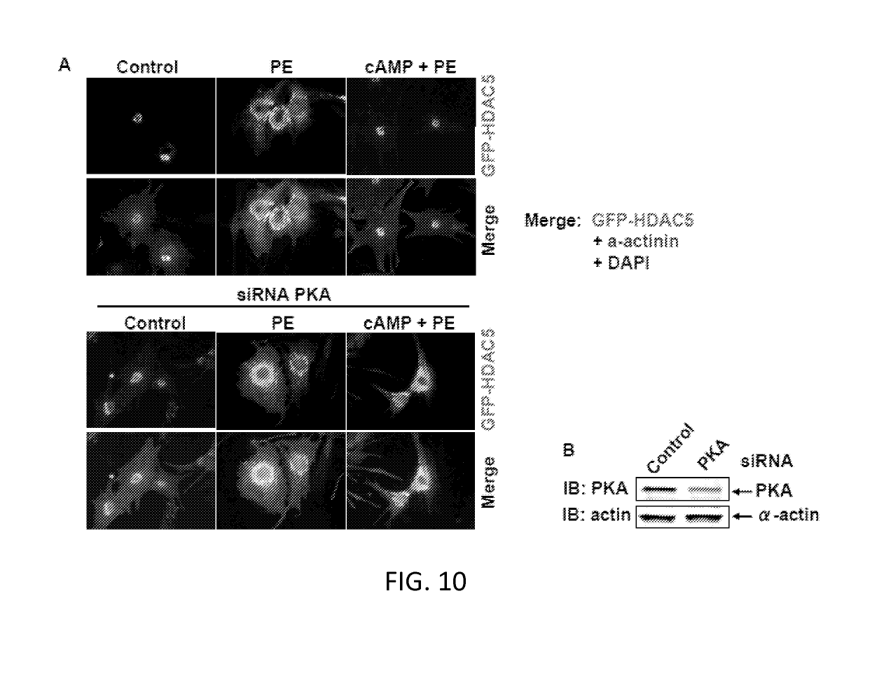

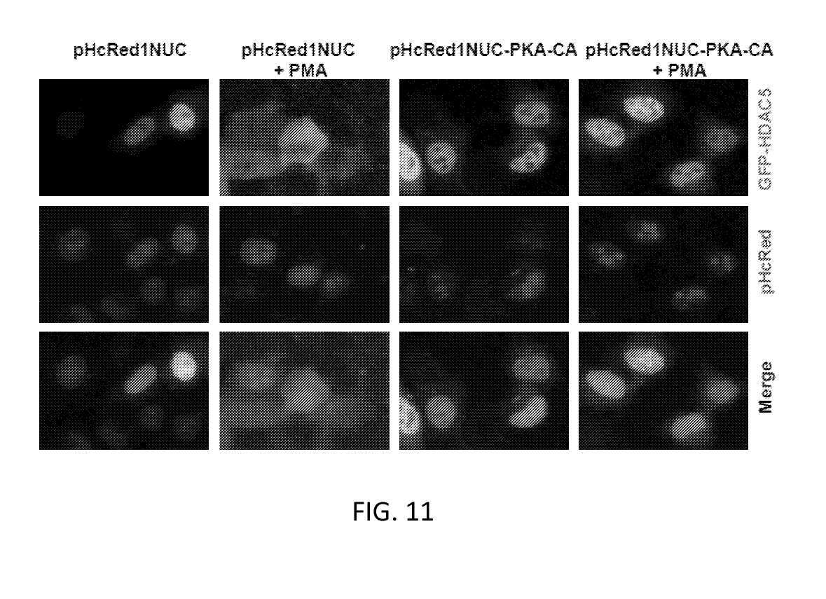

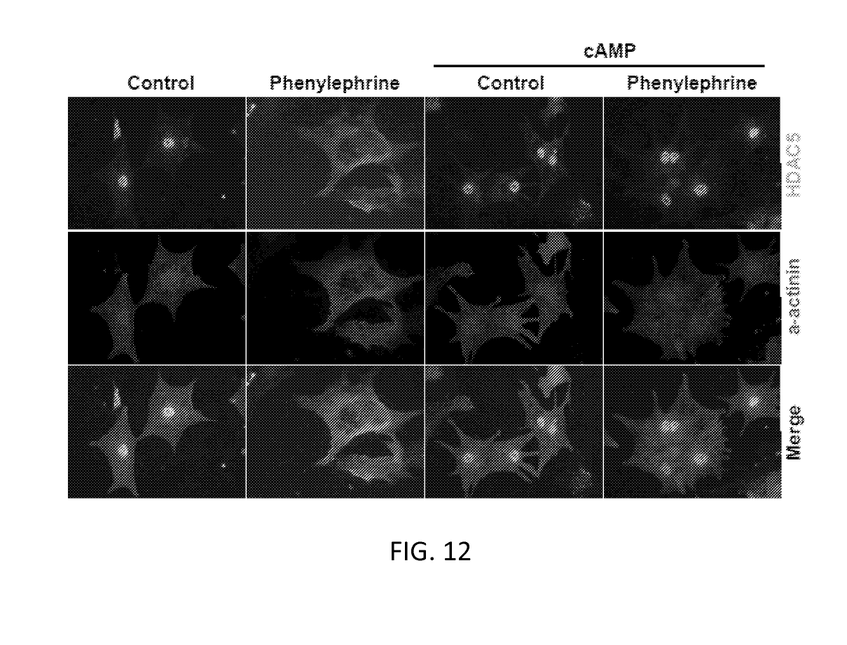

To determine whether HDAC5 nuclear export is regulated by the cAMP/PKA pathway in other type of cells, neonatal rat ventricular myocytes (NRVMs) were infected with adenovirus expressing GFP-tagged HDAC5 and then treated with forskolin, cAMP, or β-adrenergic receptor (β-AR) agonist isoproterenol for 30 min, followed by the treatment of α-adrenergic receptor agonist phenylephrine (PE, 10 μM). Forskolin, cAMP and isoproterenol markedly inhibited PE-stimulated HDAC5 nuclear export (FIGS. 1E and F). The positive staining of the myocyte marker sarcomeric α-actinin was confirmed. Similar results were observed when adult rat ventricular myocytes were used (FIG. 8). In agreement with the results observed in Cos7 cells, the inhibition of PKA by PKI and small interference RNA in NRVMs abolished the inhibitory effects of cAMP on PE-induced HDAC5 nuclear export (FIGS. 9 and 10). Since PKA-CA has long been shown to enter and exit the nucleus of cells when intracellular cAMP is raised and lowered, respectively, whether nuclear PKA could affect HDAC5 localization was studied. The transfection of nuclear-localized HcRed1-tagged PKA-CA-NLS (NLS, nuclear localization sequence) inhibited HDAC5 nuclear export (FIG. 11), suggesting that the PKA inhibitory effect on HDAC5 nuclear export could occur in the nucleus. Moreover, the same inhibitory effect of cAMP on PE-induced endogenous HDAC5 nuclear export in NRVMs was also observed (FIG. 12). Collectively, these findings reveal that cAMP/PKA signaling specifically and negatively regulates stress signal-dependent nuclear export of HDAC5.

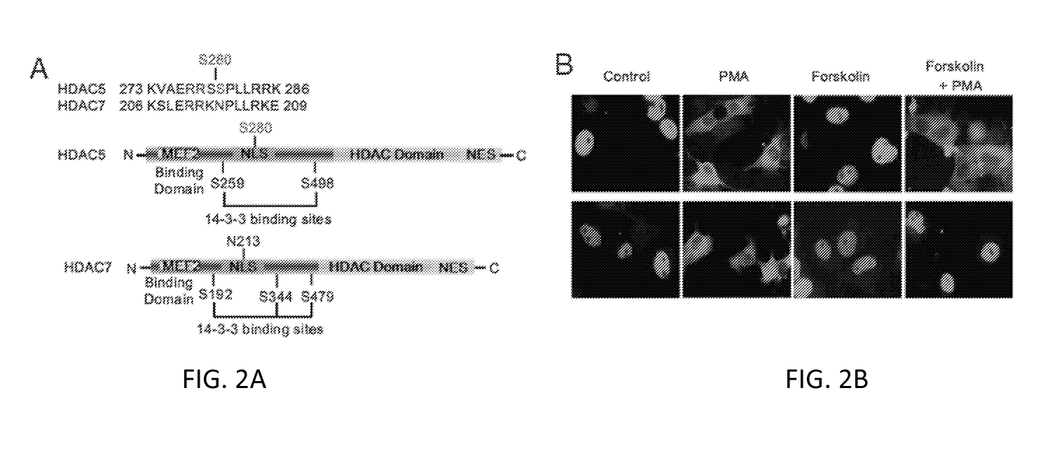

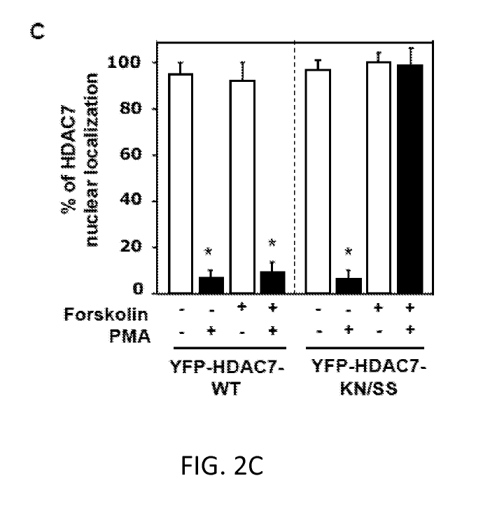

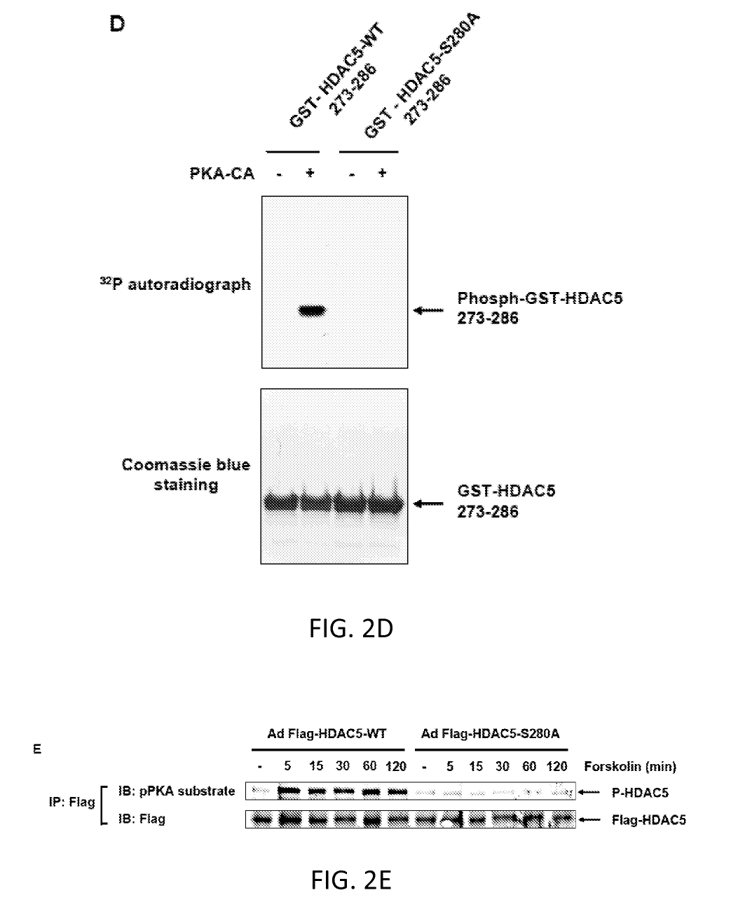

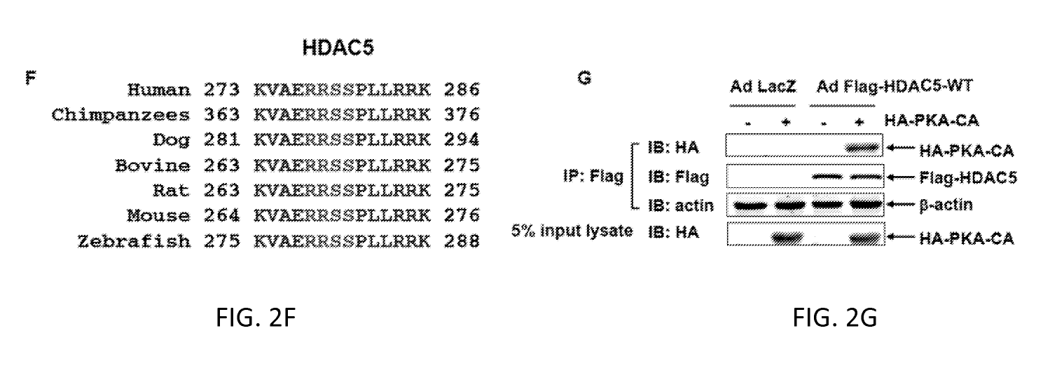

To determine whether the cAMP/PKA pathway regulates other members of class IIa HDACs, the effects forskolin/cAMP on PMA-induced nuclear export of YFP-tagged HDAC7 in Cos7 cells were examined. Interestingly, there was no inhibitory effect of cAMP/PKA on HDAC7 nuclear export (FIGS. 2B and C). Taken together, these results indicate that the cAMP/PKA pathway selectively controls nucleocytoplasmic shuttling of HDAC5 but not HDAC7.

### HDAC5 is a Novel Substrate for PKA

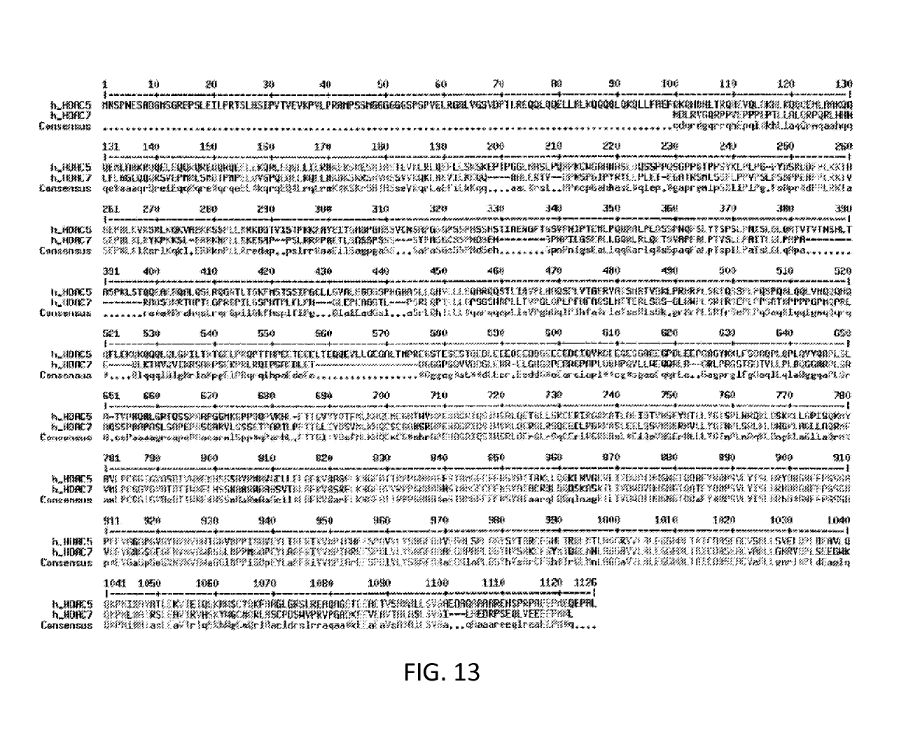

To address the mechanisms by which PKA regulates HDAC5 subcellular localization, the amino acid sequences of human HDAC5 and human HDAC7 were compared. It was noticed that a potential PKA targeting motif “RRSS” in HDAC5 is replaced by “RRKN” in HDAC7 (FIG. 2A). Both HDAC5 and HDAC7 have N-terminal MEF2 binding domain, nuclear localization sequence (NLS), and C-terminal HDAC domain and nuclear export sequence (NES). There are several conserved phosphorylation sites near the nuclear localization sequence (NLS), which are scaffolding protein 14-3-3 binding sites. The potential PKA phospho-site Serine (S) 280 in human HDAC5 is replaced with asparagine (N) 213 in human HDAC7. To determine whether the “RRSS” motif is responsible for PKA-dependent inhibition of HDAC5 nuclear export, performed site-directed mutagenesis was performed to replace “KN” in HDAC7 with “SS”. In contrast to the inability of PKA to inhibit YFP-HDAC7-WT nuclear export, it was found that nuclear export of the YFP-HDAC7-KN/SS mutant was blocked by the treatment of PKA activator forskolin (FIGS. 2B and C). Given the fact that there is only 50% identity of amino acids between HDAC5 and HDAC7 (FIG. 13), these results indicate that the “RRSS” motif is responsible for the PKA inhibitory effect on HDAC5 nuclear export.

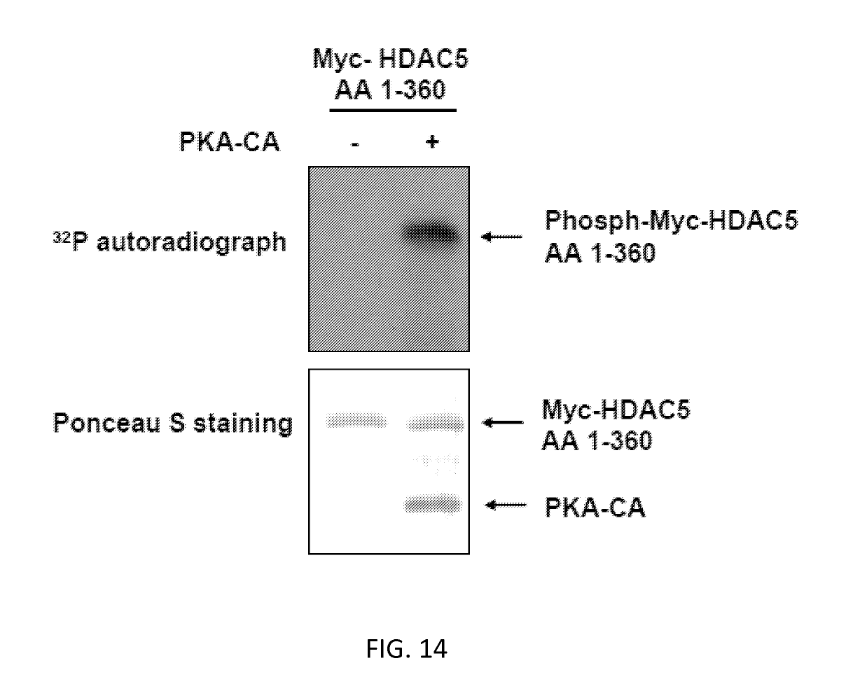

Since PKA prefers to phosphorylate the serine in the “RRXS” motif, the phosphorylation of Ser280 in HDAC5 likely confers the inhibition of HDAC5 nuclear export by PKA. To determine whether PKA is able to phosphorylate HDAC5 at the Ser280 site, an in vitro kinase assay using GST-tagged peptides containing residues 273-286 of HDAC5 wild type (WT) and S280A mutant, in which serine 280 was replaced with alanine was performed. It was observed that recombinant PKA-CA phosphorylated the GST-tagged peptide of HDAC5-WT but not the GST-tagged peptide of HDAC5-S280A mutant (FIG. 2D). Using a larger, more natural fragment of HDAC5 (1-360 amino acids, covering Ser280 and containing the entire NLS) (1), the similar phosphorylation by PKA-CA in an in vitro kinase assay (FIG. 14) was also detected. In addition, using a phospho-(Ser/Thr) PKA substrate antibody, PKA-dependent phosphorylation of full length HDAC5-WT but not HDAC5-S280A mutant in Cos7 cells (FIG. 2E) was observed. Notably, the PKA phosphorylation site in HDAC5 is evolutionally conserved from zebrafish to human (FIG. 2F). Moreover, the association between PKA and HDAC5 in Cos7 cells was also detected by co-immunoprecipitation (FIG. 2G). Taken together, these results demonstrate for the first time that HDAC5 is a novel substrate of PKA.

Phosphorylation of HDAC5 on Ser280 Mediates the Inhibition of HDAC5 Nuclear Export by the cAMP/PKA Pathway

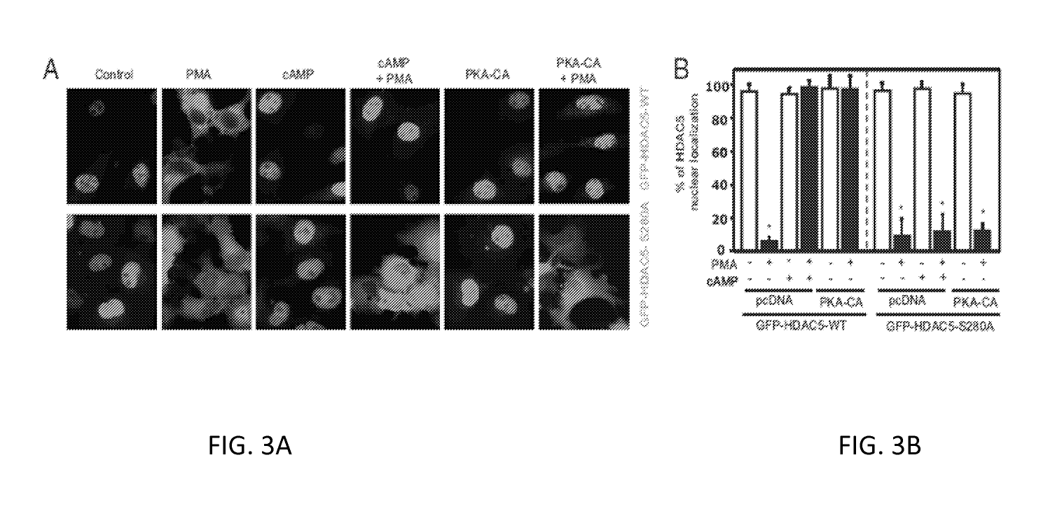

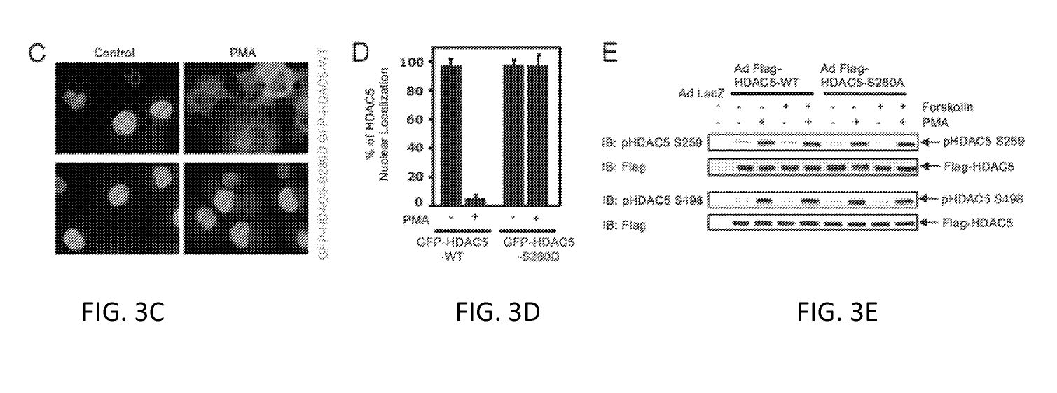

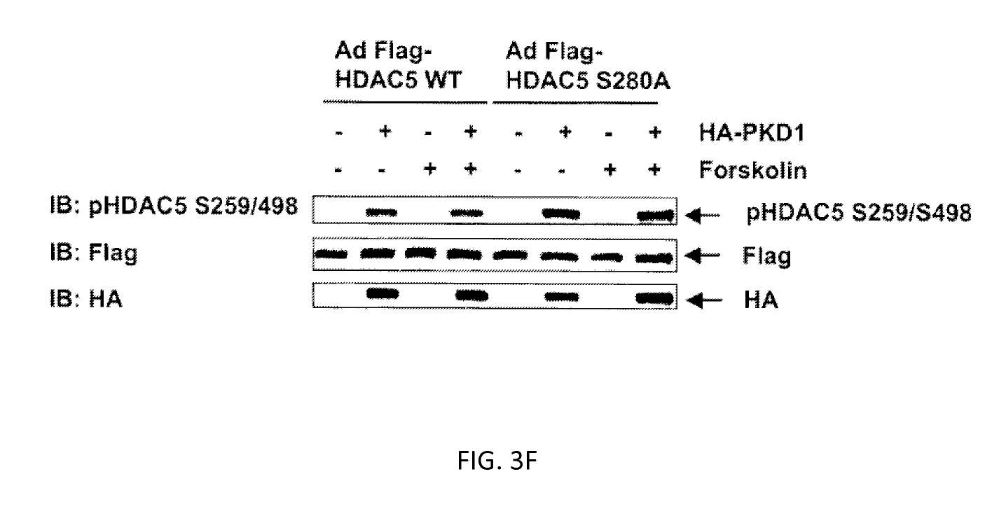

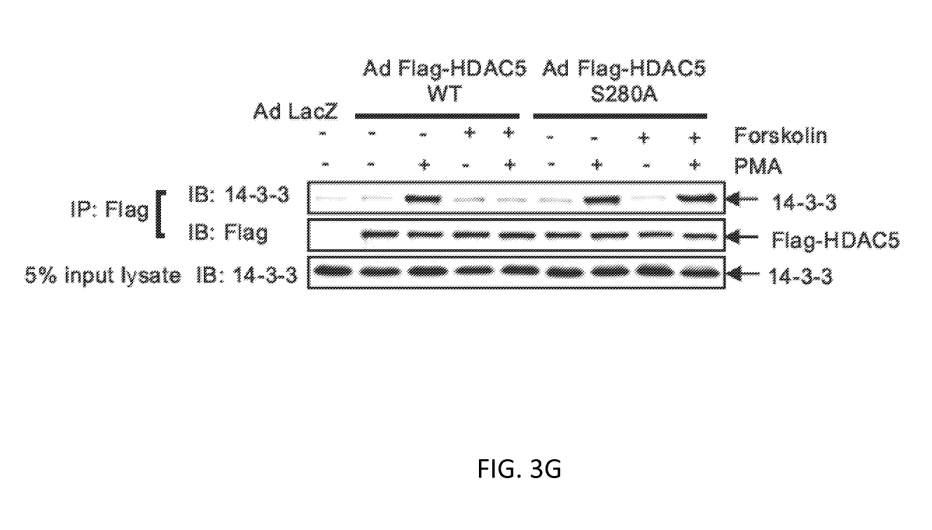

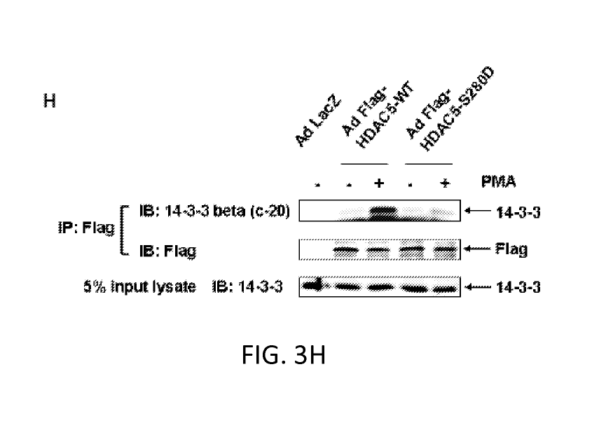

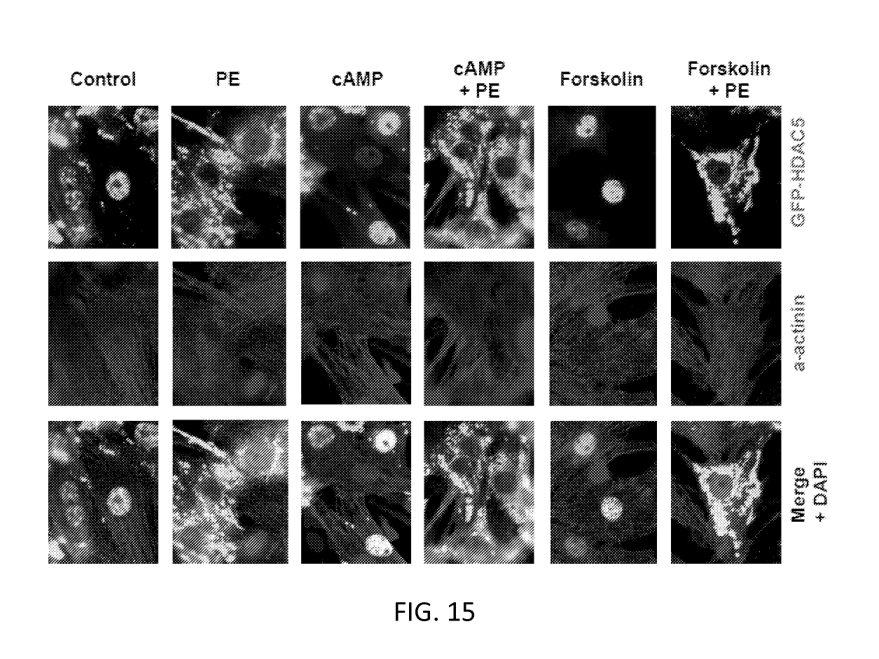

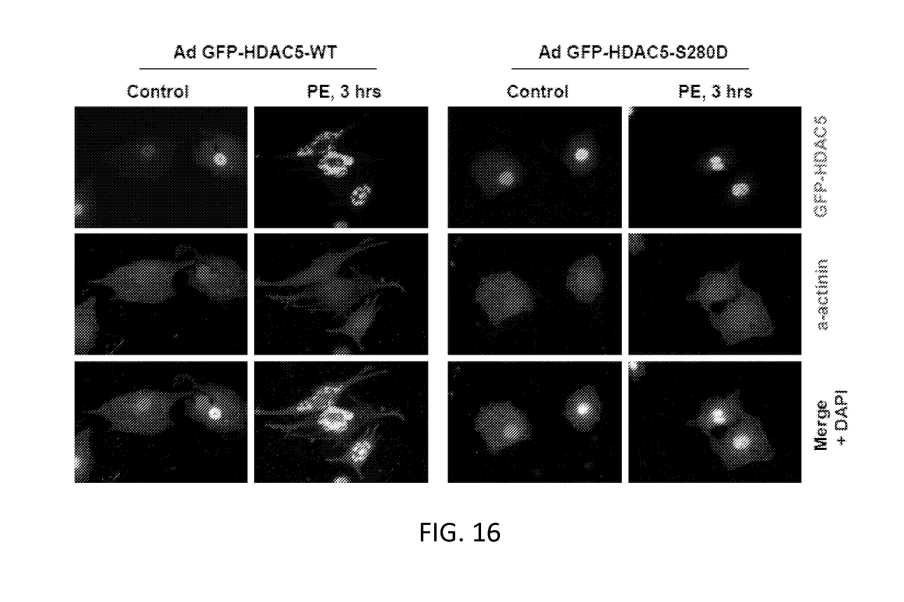

To determine whether PKA-dependent phosphorylation of HDAC5 on Ser280 mediates its inhibitory effect on HDAC5 nuclear export, the effect of PKA on subcellular localization of the HDAC5-S280A mutant in Cos7 cells was studied. In contrast to the inhibition of PKA on GFP-HDAC5-WT nuclear export, there was no inhibitory effect of PKA on PMA-induced nuclear export of GFP-HDAC5-S280A (FIGS. 3A and B). Similar results were observed in cardiomyocytes infected with adenoviral GFP-HDAC5-S280A (FIG. 15). These results indicate that Ser280 is necessary for PKA-mediated inhibition of HDAC5 nuclear export. To ask whether the phosphorylation of Ser280 is sufficient to mediate PKA inhibition of HDAC5 nuclear export, a GFP-tagged HDAC5-S280D mutant was generated, in which serine 280 was replaced with aspartic acid to mimic PKA-dependent phosphorylation. When this protein was expressed in Cos7 cells, it was found that the GFP-HDAC5-S280D mutant is resistant to nuclear exclusion in response to PMA (FIGS. 3C and D). Similar results were observed when cardiomyocytes were infected with adenoviral GFP-HDAC5-S280D (FIG. 16). Collectively, these results show that PKA-dependent phosphorylation on Ser280 mediates nuclear retention of HDAC5.

### PKA-Dependent Phosphorylation of HDAC5 Impairs the Association of HDAC5 and 14-3-3

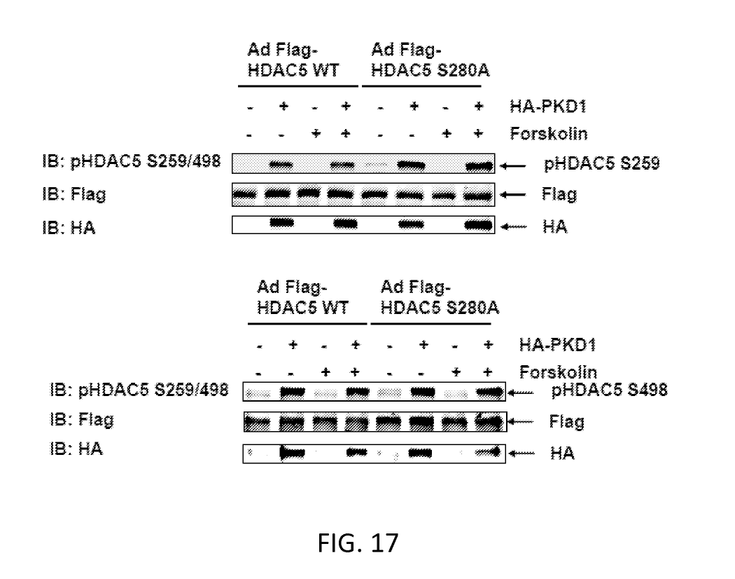

To determine the mechanisms by which PKA-dependent phosphorylation of HDAC5 controls its subcellular localization, whether PKA affected HDAC5 phosphorylation on serine 259 and 498 residues that are responsible for the recruitment of 14-3-3 proteins and subsequent nuclear export of HDAC5 was examined. Western blot analysis showed that PKA activators did not affect HDAC5 phosphorylation at Ser259 and Ser498 sites in response to PMA (FIGS. 3E and F). Similar data were obtained in experiments when Cos7 cells were co-transfected with constitutively active PKD1-S738E/S742E mutant and Flag-HDAC5-WT (FIG. 17). These results indicate that there is no crosstalk between these two functionally distinct phosphorylation events. Next, whether PKA affected the recruitment of 14-3-3 proteins by HDAC5 was examined. Co-immunprecipitation experiments showed that PKA stimulation markedly attenuated PMA-induced the association of HDAC5 and 14-3-3 proteins (FIG. 3G). However, there was no inhibitory effect of PKA on the association of HDAC5-S280A mutant and 14-3-3 proteins (FIG. 3G). Furthermore, HDAC5-S280D mutant prevented its interaction with 14-3-3 proteins (FIG. 3H). These results demonstrate that PKA-dependent HDAC5 phosphorylation at Ser280 interferes with the interaction of HDAC5 and 14-3-3, resulting in the inhibition of HDAC5 nuclear export. Since the Ser280 residue lies within the region of the NLS and between two 14-3-3 binding sites, Ser259 and Ser498 (FIG. 2A), it is likely that PKA-dependent Ser280 phosphorylation can change the conformation of HDAC5 and hence block 14-3-3 binding which results in HDAC5 nuclear retention.

### PKA-Dependent HDAC5 Phosphorylation and Nuclear Retention Represses MEF2-Dependent Gene Transcription and Cardiac Fetal Gene Expression

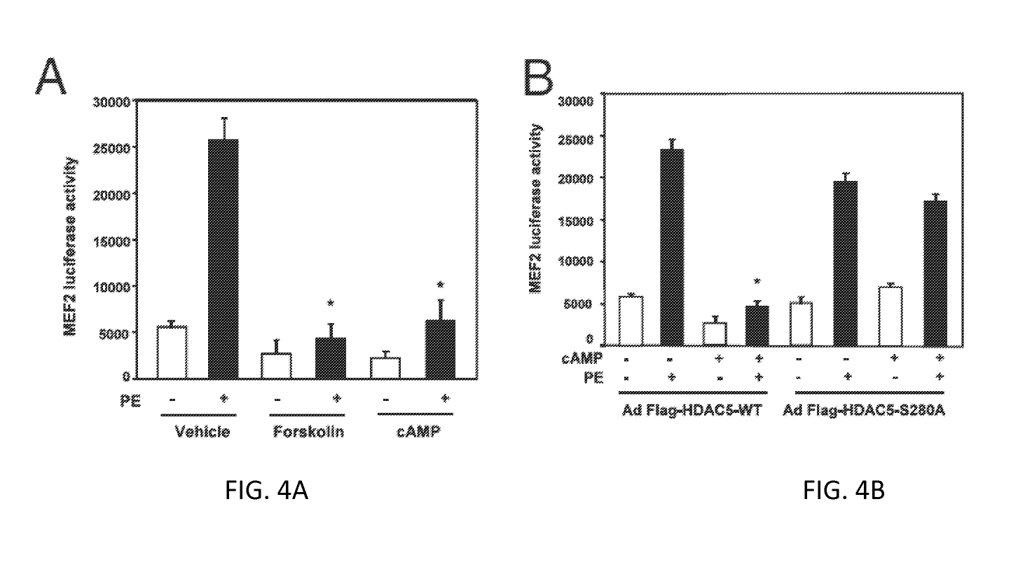

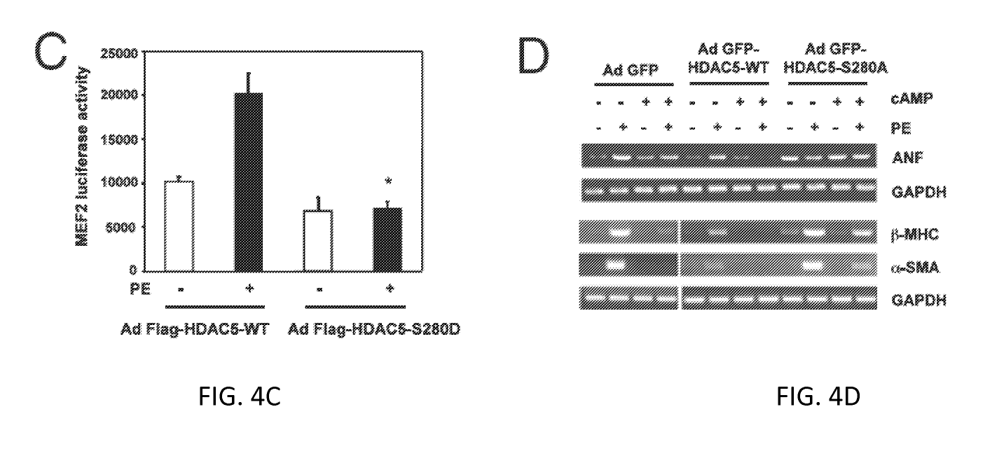

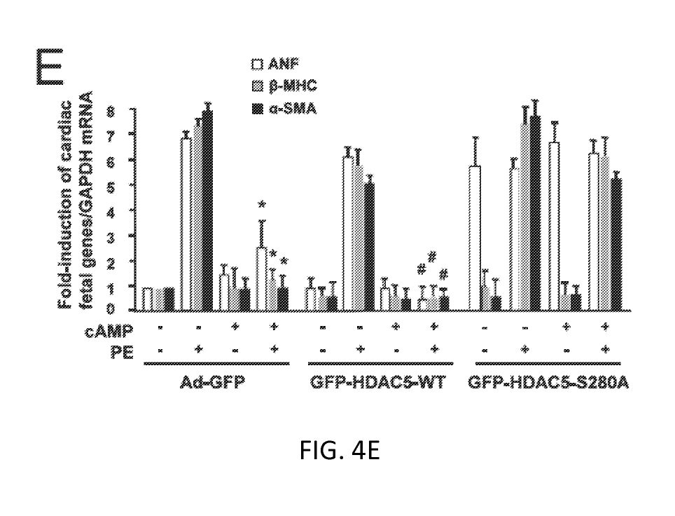

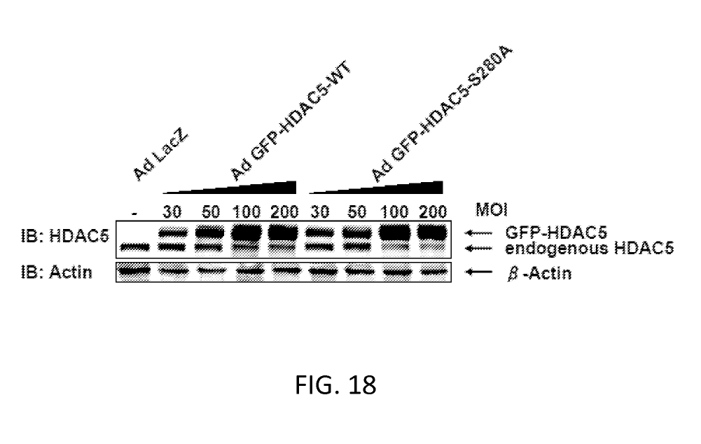

HDAC5 is highly expressed in the heart, skeletal muscle, vasculature and brain. HDAC5 binds and represses MEF2 transcriptional factor to silence MFE2-dependent gene transcriptional programs that control cell differentiation and cell growth. To address the biological role of PKA-dependent HDAC5 phosphorylation, the effects of the PKA activators and the HDAC5 mutants on MEF2 transcriptional activity were examined. A luciferase reporter containing 3×MEF2 sites was utilized to assess MEF2 transcriptional activity. In NRVMs infected with adenovirus expressing GFP alone and GFP-tagged HDAC5-WT, cAMP significantly inhibited PE-stimulated MEF2 transcriptional activity (FIG. 4A). Interestingly, co-infection of adenoviral MEF2 luciferase construct along with adenoviral GFP-HADC5-S280A abolished the decrease in MEF2 transcriptional activation by cAMP (FIG. 4B). Of note, overexpression of HDAC5-WT or mutant dose-dependently decreases endogenous HDAC5 expression (see supporting information FIG. 18). In contrast to that with the HDAC5-S280A mutant, infection with adenoviruses expressing GFP-HADC5-S280D blocked PE-stimulated MEF2 transcriptional activity (FIG. 4C). These results indicate that cAMP/PKA-dependent phosphorylation and nuclear accumulation of HDAC5 negatively regulates MEF2 transcriptional activity.

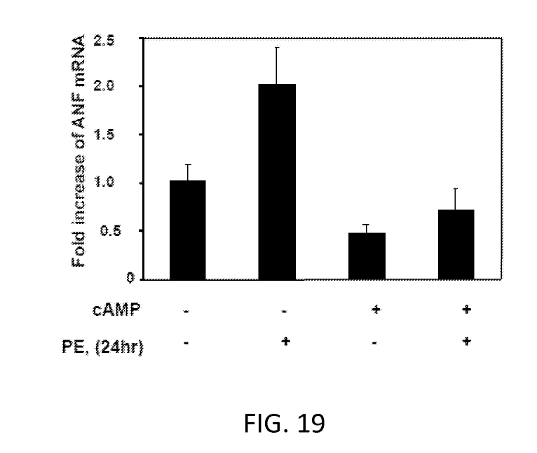

To determine the role of PKA-induced HDAC5 phosphorylation on gene expression in NRVMs, RT-PCR and real-time PCR were used to study the expression of several cardiac fetal genes (hypertrophic marker genes) including atrial natriuretic factor (ANF), β-myosin heavy chain (β-MHC), and α-skeletal muscle actin (α-SMA) (5). Treatment with PE significantly increased expression of ANF, β-MHC and α-SMA in cells infected with adenoviruses expressing GFP-tagged HDAC5-WT; cAMP treatment blocked this elevated gene expression (FIGS. 4D and E, and FIG. 19). In contrast, when cells were infected with adenoviruses expressing an HDAC5-S280A mutant, the PKA inhibitory effect was markedly attenuated (FIGS. 4D and E). These data indicate that cAMP/PKA-dependent phosphorylation and nuclear accumulation of HDAC5 negatively regulates cardiac fetal gene expression.

### PKA-Dependent HDAC5 Phosphorylation and Nuclear Retention Inhibits Cardiomyocyte Hypertrophy

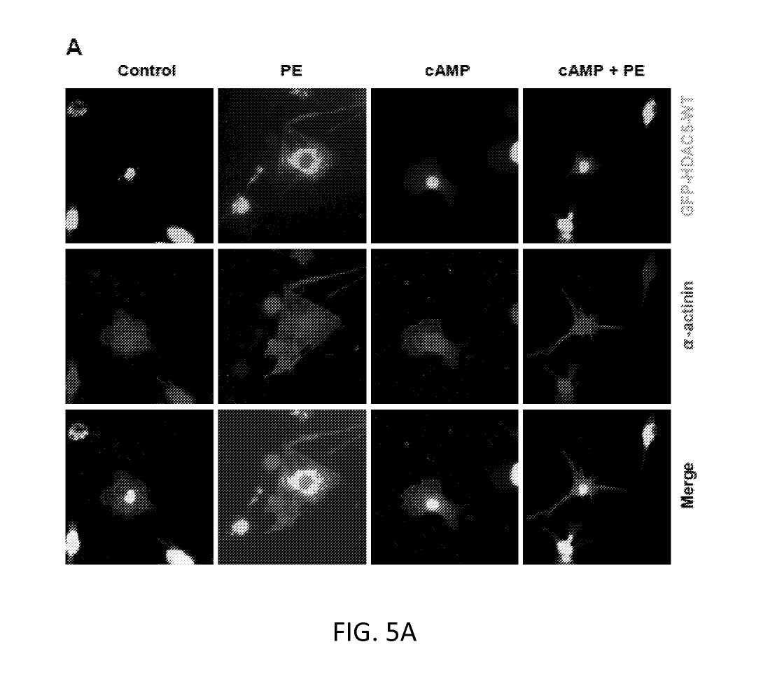

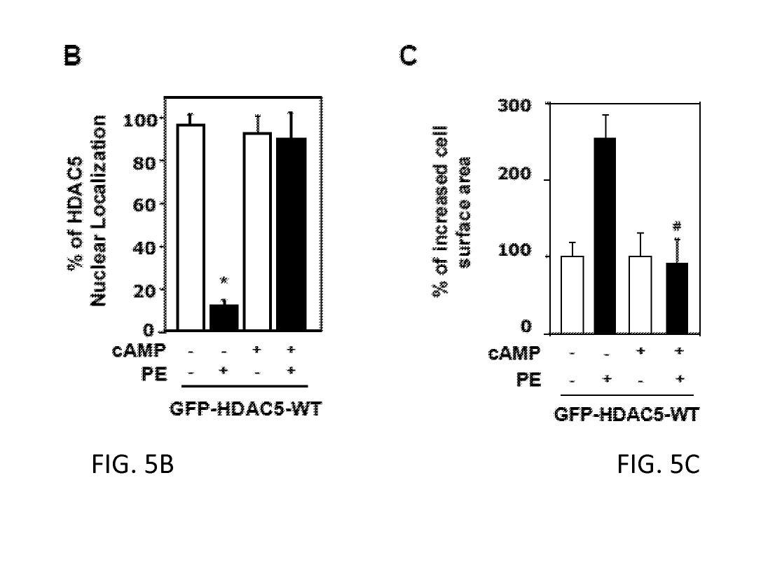

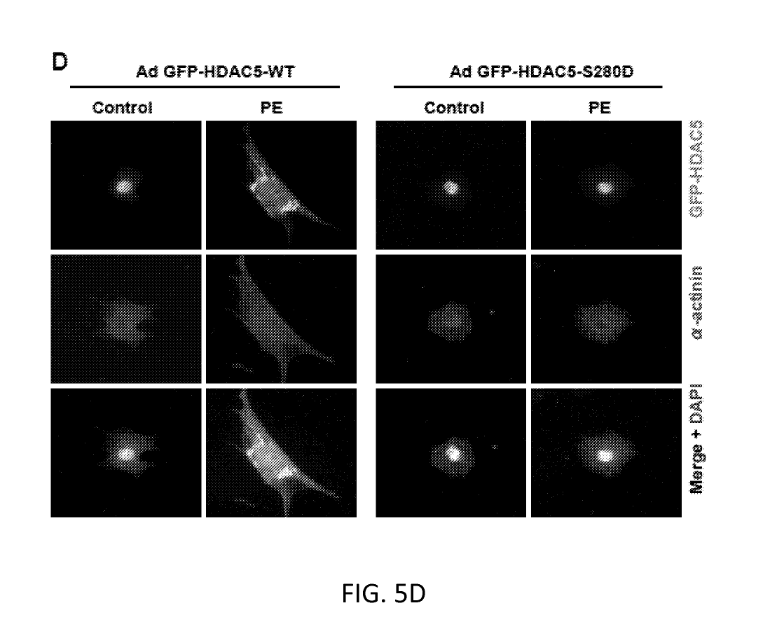

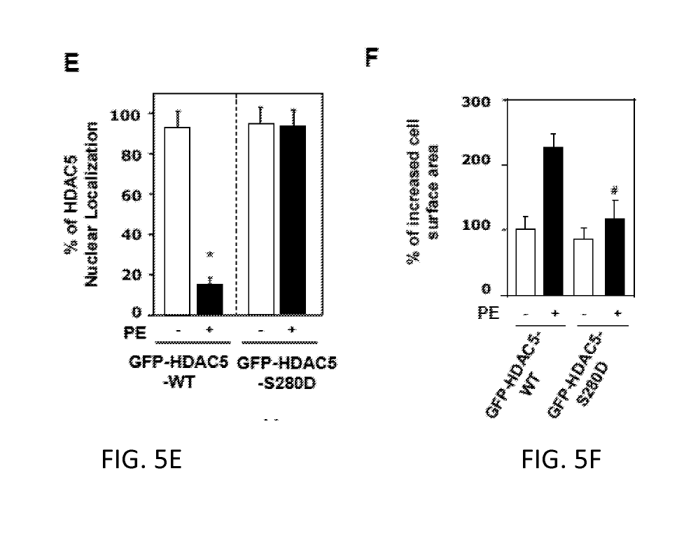

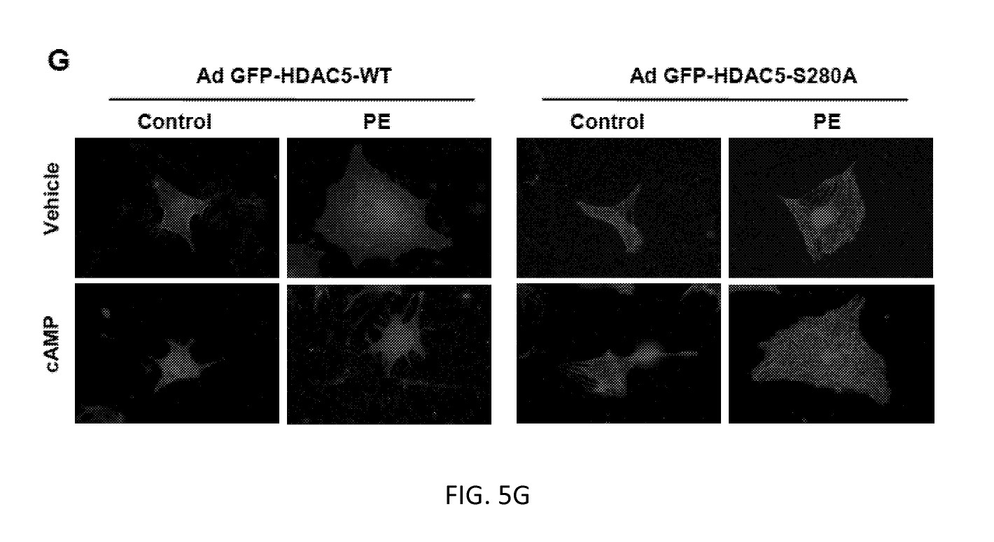

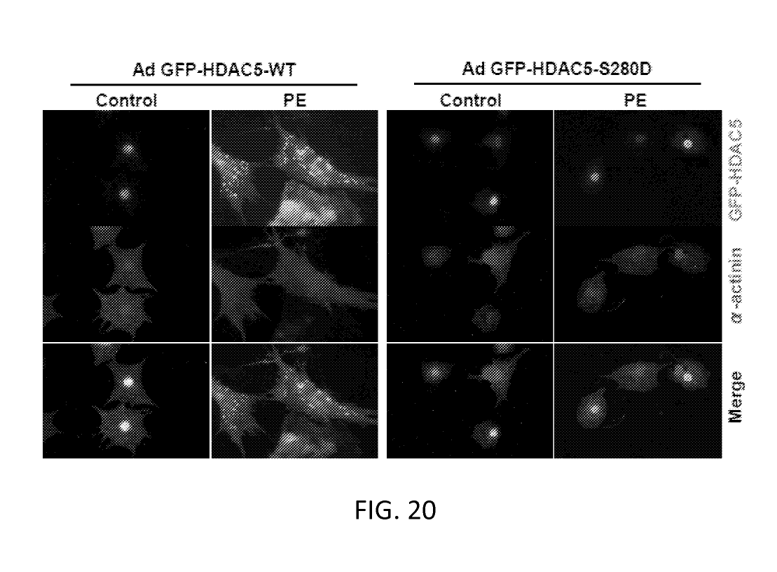

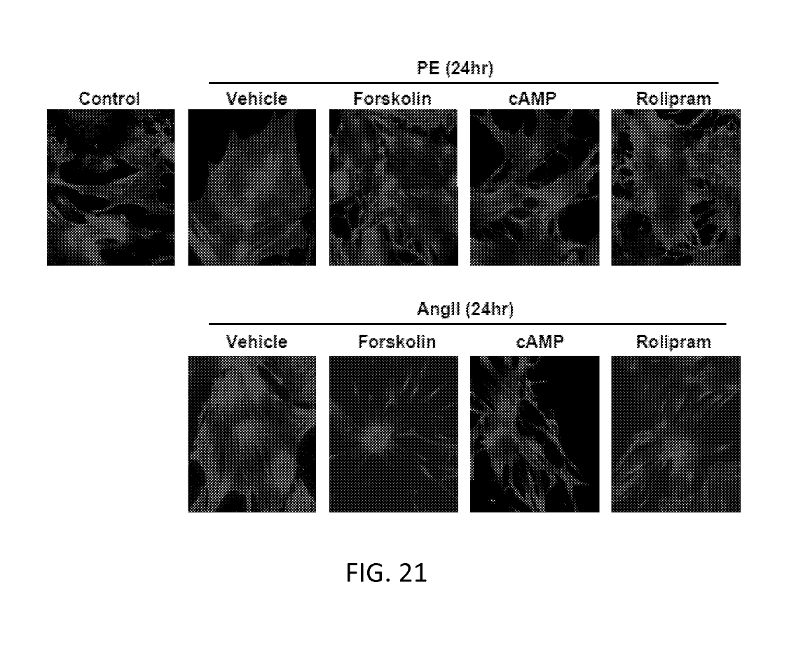

Cardiac fetal gene expression contributes to cardiac growth and hypertrophy. A major feature of the hypertrophic response of cardiomyocytes is a pronounced sarcomeric rearrangement and enlargement of cell size that can be detected by immunostaining with α-actinin antibody. Thus, the effect of the PKA/HDAC5 pathway on cardiomyocyte hypertrophy was studied. NRVMs were infected with adenoviruses expressing GFP-tagged HDAC5-WT for 24 hours, and then treated with cAMP for 30 min, followed by PE for 24 hours. It was found that PE treatment leads to increased cell size and HDAC5 nuclear export (FIG. 5A-C). However, the addition of cAMP decreased the size of cardiomyocytes and HDAC5 was prevented from translocating from the nucleus to the cytoplasm (FIG. 5A-C). Unlike GFP-tagged HDAC5-WT-infected cells, the cells infected with adenoviruses expressing GFP-tagged HDAC5-S280D mutant showed the nuclear accumulation of HDAC5-S280D and reduced cell size after PE treatment (FIG. 5D-F and FIG. 20). Furthermore, infection of GFP-tagged HDAC5-S280A attenuated the inhibitory effect of PKA on PE-stimulated an increase of cardiomyocyte size (FIGS. 5G and H). Similar effects were observed when NRVMs were treated with angiotensin II instead of PE (FIG. 21). These results show that PKA-dependent phosphorylation and nuclear retention of HDAC5 inhibits cardiomyocyte hypertrophy.

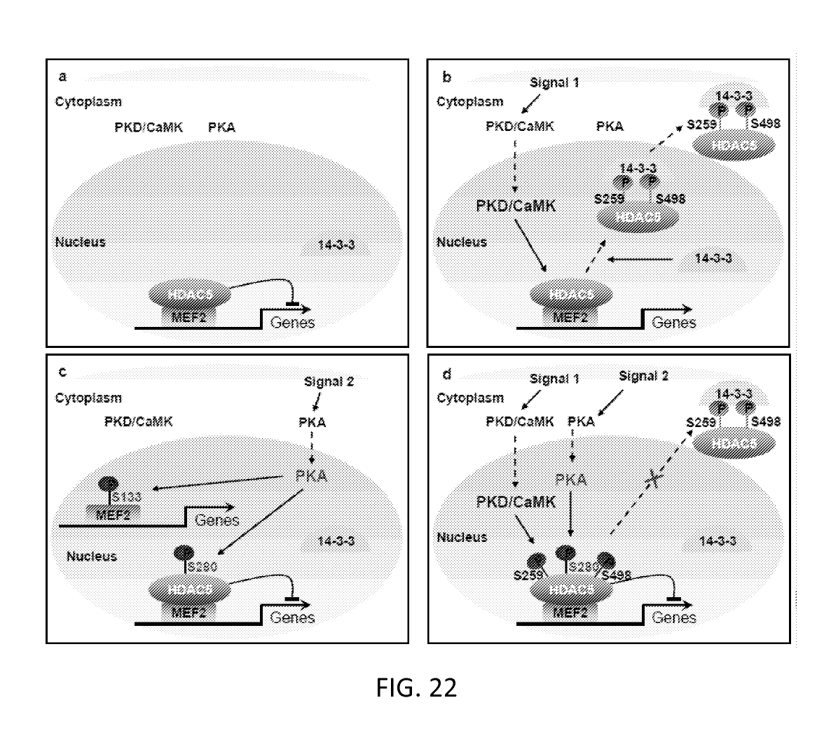

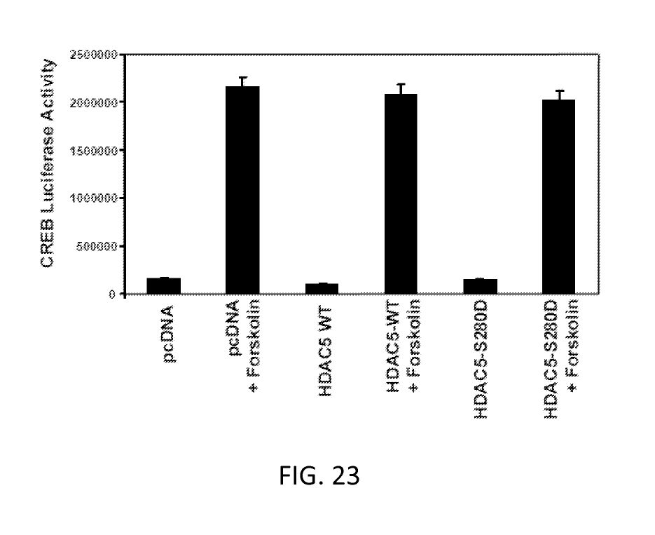

The regulation of HDAC5 nuclear accumulation by PKA provides a mechanism for cell type-specific responses to extracellular stimulation. In contrast to the positive regulation of CREB-dependent gene transcription by the cAMP/PKA pathway, these studies show that PKA phosphorylates HDAC5 and blocks its nuclear export in cells, which negatively regulates MEF2-dependent gene expression and cardiomyocyte hypertrophy in response to stress signals (FIG. 5I, and FIG. 22). Interestingly, these two pathways are distinctly regulated by PKA because HDAC5 has no inhibitory effect on CREB transcriptional activity (FIG. 23). Given the important regulatory functions of cAMP/PKA and HDAC5/MEF2 signaling in cell differentiation, proliferation, morphogenesis, survival and apoptosis in various tissues and systems, the identification of the molecular link between both has broad implications for the regulation of a wide range of biological functions and human diseases such as cardiomyopathy, neuronal disease and metabolic disorders.

1. A method of inhibiting HDAC5 activity in a cell, comprising contacting the cell with an agent that increases cAMP/PKA/HDAC5 pathway activity in the cell, wherein the cell is subjected to stress.

2. The method of claim 1, wherein the HDAC 5 activity is selected from the group consisting of nuclear export and interaction between HDAC5 and 14-3-3 protein.

3. (canceled)

4. The method of claim 1, wherein inhibition of HDAC5 activity represses MEF2-dependent gene expression or inhibits cardiac fetal gene expression.

5. (canceled)

6. The method of claim 1, wherein the agent is selected from the group consisting of a protein kinase A activator and an agent that increases cAMP levels.

7. The method of claim 6, wherein the protein kinase A activator is selected from the group consisting of forskolin and cAMP.

8. (canceled)

9. The method of claim 6, wherein the agent that increases cAMP levels is selected from the group consisting of a cAMP-specific PDE IV inhibitor, a Gₛ activator, a PDE II inhibitor and a beta-adrenergic agonist.

10. The method of claim 1, wherein the cell is a myocyte.

11.-12. (canceled)

13. A method of preventing cardiac myopathy or of preventing or delaying the occurrence of heart failure in a subject, comprising administering to the subject an agent that increases cAMP/PKA/HDAC5 pathway activity.

14. The method of claim 13, wherein the agent is a protein kinase A activator or an agent that increases cAMP levels.

15. The method of claim 14, wherein the protein kinase A activator is selected from the group consisting of forskolin and cAMP.

16. (canceled)

17. The method of claim 14, wherein the agent that increases cAMP levels is selected from the group consisting of a cAMP-specific PDE IV inhibitor, a Gₛ activator, a PDE II inhibitor and a beta-adrenergic agonist.

18.-22. (canceled)

23. A method of treating heart failure comprising administering to the subject an agent that decreases cAMP/PKA/HDACS pathway activity.

24. The method of claim 23, wherein the agent that decreases cAMP/PKA/HDAC5 pathway activity is selected from the group consisting of an agent that inhibits binding of HDAC5 to PKA, an agent that inhibits phosphorylation of HDAC5 at Ser280 by PKA, an antibody that selectively binds the PKA binding site of HDAC5, an antibody that selectively binds the HDAC5 binding site of PKA, a peptide.

25.-27. (canceled)

28. The method of claim 24, wherein the peptide comprises the amino acid sequence of SEQ ID NO: 2 or a fragment thereof.

29. A method of treating heart failure in a subject comprising administering to the subject an agent that inhibits HDAC5-mediated transcriptional repression of MEF2.

30. The method of claim 29, wherein the agent is selected from the group consisting of an anti-HDAC5 antibody, an HDAC5 inhibitor, a peptide that inhibits HDAC-5 mediated transcriptional repression of MEF2, an antisense nucleic acid that inhibits HDAC5 mediated transcriptional repression of MEF2, an siRNA that inhibits HDAC5 mediated transcriptional repression of MEF2, or a microRNA that inhibits HDAC5 mediated transcriptional repression of MEF2.

31.-33. (canceled)

34. A method of identifying a compound that inhibits cardiac myopathy via the cAMP/PKA/HDAC5 pathway comprising:
a) contacting a cardiomyocyte with a test compound, wherein the cardiac myocyte is subjected to stress; and
b) measuring phosphorylation of HDAC5 at Ser280, wherein an increase in phosphorylation indicates that the test compound inhibits cardiac myopathy via the cAMP/PKA/HDAC5 pathway.

35. The method of claim 34, further comprising one or more of the steps of
a) measuring the size of the cardiomyocyte, a decrease in size indicating that the test compound inhibits cardiac myopathy and
b) measuring nuclear export of HDAC5, wherein a decrease in nuclear export indicates that the test compound inhibits cardiac myopathy.

36. (canceled)

37. A method of identifying a compound that inhibits cardiac myopathy via the cAMP/PKA/HDAC5 pathway comprising:
a) contacting a cardiomyocyte with a test compound, wherein the cardiac myocyte is subjected to stress; and
b) measuring the association of HDAC5 with 14-3-3 protein, a decrease in the association between HDAC5 and 14-3-3 indicating that the test compound inhibits cardiac myopathy via the cAMP/PKA/HDAC5 pathway.

38. The method of claim 37, further comprising one or more of the steps of
a) measuring the size of the cardiomyocyte, a decrease in size indicating that the test compound inhibits cardiac myopathy, and
b) measuring nuclear export of HDAC5, wherein a decrease in nuclear export indicates that the test compound inhibits cardiac myopathy.

39. (canceled)

40. A method of identifying a compound that inhibits the binding of HDAC5 to PKA comprising:
a) contacting HDAC5 with a test compound in the presence of PKA; and
b) detecting binding of HDAC5 to PKA, a decrease in binding compared to a control indicating that the test compound inhibits binding of HDAC5 to PKA.

41. A method of identifying a compound that inhibits binding of HDAC5 to PKA comprising:
a) contacting a cell that comprises HDAC5, PKA and a construct containing an MEF2 regulatory region operatively linked to a nucleic acid encoding luciferase with a test compound; and
b) detecting the level of luciferase activity, an increase in luciferase activity indicating that the compound inhibits binding of HDAC5 to PKA.

42. A method of identifying a compound that inhibits HDAC5-mediated transcriptional repression of MEF2 comprising:
a) contacting a cell with a test compound; and
b) detecting the level of expression of MEF2-dependent gene(s), wherein an increase in expression of MEF2 dependent genes as comparted to control indicates that the test compound is a compound that inhibits HDAC5-mediated transcriptional repression of MEF2.


SEQUENCE LISTING (21 sequences)

SEQ ID NO: 1 - protein, 20 aa, Artificial Sequence [sequence omitted]

SEQ ID NO: 2 - protein, 14 aa, Artificial Sequence
  Synthetic Construct
KVAERRSSPLLRRK

SEQ ID NO: 3 - DNA, 30 bp, Artificial Sequence
  Synthetic Construct
gctgagcggagaagcgctcccctcctgcgt

SEQ ID NO: 4 - DNA, 30 bp, Artificial Sequence
  Synthetic Construct
acgcaggaggggagcgcttctccgctcagc

SEQ ID NO: 5 - DNA, 30 bp, Artificial Sequence
  Synthetic construct
gctgagcggagaagcgatcccctcctgcgt

SEQ ID NO: 6 - DNA, 30 bp, Artificial Sequence
  Synthetic Construct
acgcaggaggggatcgcttctccgctcagc

SEQ ID NO: 7 - DNA, 32 bp, Artificial Sequence
  Synthetic construct
cctggagagacgcagcagtcccctgctcagga

SEQ ID NO: 8 - DNA, 32 bp, Artificial Sequence
  Synthetic Construct
tcctgagcaggggactgctgcgtctctccagg

SEQ ID NO: 9 - DNA, 64 bp, Artificial Sequence
  Synthetic Construct
gctagtccggaatgtacccatacgatgttccagattacgctatgggcaacgccgccgccgccaa

SEQ ID NO: 10 - DNA, 32 bp, Artificial Sequence
  Synthetic Construct
gccctcgagcaaactcagtaaactccttgcca

SEQ ID NO: 11 - DNA, 20 bp, Artificial Sequence
  Synthetic Construct
atgggctccttctccatcac

SEQ ID NO: 12 - DNA, 20 bp, Artificial Sequence
  Synthetic Construct
atcttcggtaccggaagctg

SEQ ID NO: 13 - DNA, 20 bp, Artificial Sequence
  Synthetic Construct
actgggacgacatggaaaag

SEQ ID NO: 14 - DNA, 20 bp, Artificial Sequence
  Synthetic Construct
catctccagagtccagcaca

SEQ ID NO: 15 - DNA, 20 bp, Artificial Sequence
  Synthetic Construct
cctcgcaatatcaagggaaa

SEQ ID NO: 16 - DNA, 20 bp, Artificial Sequence
  Synthetic Construct
tacaggtgcatcagctccag

SEQ ID NO: 17 - DNA, 18 bp, Artificial Sequence
  Synthetic Constuct
cgatgctggcgctgagta

SEQ ID NO: 18 - DNA, 20 bp, Artificial Sequence
  Synthetic Construct
cgttcagctcagggatgacc

SEQ ID NO: 19 - protein, 1123 aa, Homo sapiens
MNSPNESADGMSGREPSLEILPRTSLHSIPVTVEVKPVLPRAMPSSMGGGGGGSPSPVELRGALVGSVDPTLREQQLQQELLALKQQQQLQKQLLFAEFQKQHDHLTRQHEVQLQKHLKQQQEMLAAKQQQEMLAAKRQQELEQQRQREQQRQEELEKQRLEQQLLILRNKEKSKESAIASTEVKLRLQEFLLSKSKEPTPGGLNHSLPQHPKCWGAHHASLDQSSPPQSGPPGTPPSYKLPLPGPYDSRDDFPLRKTASEPNLKVRSRLKQKVAERRSSPLLRRKDGTVISTFKKRAVEITGAGPGASSVCNSAPGSGPSSPNSSHSTIAENGFTGSVPNIPTEMLPQHRALPLDSSPNQFSLYTSPSLPNISLGLQATVTVTNSHLTASPKLSTQQEAERQALQSLRQGGTLTGKFMSTSSIPGCLLGVALEGDGSPHGHASLLQHVLLLEQARQQSTLIAVPLHGQSPLVTGERVATSMRTVGKLPRHRPLSRTQSSPLPQSPQALQQLVMQQQHQQFLEKQKQQQLQLGKILTKTGELPRQPTTHPEETEEELTEQQEVLLGEGALTMPREGSTESESTQEDLEEEDEEDDGEEEEDCIQVKDEEGESGAEEGPDLEEPGAGYKKLFSDAQPLQPLQVYQAPLSLATVPHQALGRTQSSPAAPGGMKSPPDQPVKHLFTTGVVYDTFMLKHQCMCGNTHVHPEHAGRIQSIWSRLQETGLLSKCERIRGRKATLDEIQTVHSEYHTLLYGTSPLNRQKLDSKKLLGPISQKMYAVLPCGGIGVDSDTVWNEMHSSSAVRMAVGCLLELAFKVAAGELKNGFAIIRPPGHHAEESTAMGFCFFNSVAITAKLLQQKLNVGKVLIVDWDIHHGNGTQQAFYNDPSVLYISLHRYDNGNFFPGSGAPEEVGGGPGVGYNVNVAWTGGVDPPIGDVEYLTAFRTVVMPIAHEFSPDVVLVSAGFDAVEGHLSPLGGYSVTARCFGHLTRQLMTLAGGRVVLALEGGHDLTAICDASEACVSALLSVELQPLDEAVLQQKPNINAVATLEKVIEIQSKHWSCVQKFAAGLGRSLREAQAGETEEAETVSAMALLSVGAEQAQAAAAREHSPRPAEEPMEQEPAL

SEQ ID NO: 20 - protein, 952 aa, Homo sapiens
MDLRVGQRPPVEPPPEPTLLALQRPQRLHHHLFLAGLQQQRSVEPMRLSMDTPMPELQVGPQEQELRQLLHKDKSKRSAVASSVVKQKLAEVILKKQQAALERTVHPNSPGIPYRTLEPLETEGATRSMLSSFLPPVPSLPSDPPEHFPLRKTVSEPNLKLRYKPKKSLERRKNPLLRKESAPPSLRRRPAETLGDSSPSSSSTPASGCSSPNDSEHGPNPILGSEALLGQRLRLQETSVAPFALPTVSLLPAITLGLPAPARADSDRRTHPTLGPRGPILGSPHTPLFLPHGLEPEAGGTLPSRLQPILLLDPSGSHAPLLTVPGLGPLPFHFAQSLMTTERLSGSGLHWPLSRTRSEPLPPSATAPPPPGPMQPRLEQLKTHVQVIKRSAKPSEKPRLRQIPSAEDLETDGGGPGQVVDDGLEHRELGHGQPEARGPAPLQQHPQVLLWEQQRLAGRLPRGSTGDTVLLPLAQGGHRPLSRAQSSPAAPASLSAPEPASQARVLSSSETPARTLPFTTGLIYDSVMLKHQCSCGDNSRHPEHAGRIQSIWSRLQERGLRSQCECLRGRKASLEELQSVHSERHVLLYGTNPLSRLKLDNGKLAGLLAQRMFVMLPCGGVGVDTDTIWNELHSSNAARWAAGSVTDLAFKVASRELKNGFAVVRPPGHHADHSTAMGFCFFNSVAIACRQLQQQSKASKILIVDWDVHHGNGTQQTFYQDPSVLYISLHRHDDGNFFPGSGAVDEVGAGSGEGFNVNVAWAGGLDPPMGDPEYLAAFRIVVMPIAREFSPDLVLVSAGFDAAEGHPAPLGGYHVSAKCFGYMTQQLMNLAGGAVVLALEGGHDLTAICDASEACVAALLGNRVDPLSEEGWKQKPNLNAIRSLEAVIRVHSKYWGCMQRLASCPDSWVPRVPGADKEEVEAVTALASLSVGILAEDRPSEQLVEEEEPMNL

SEQ ID NO: 21 - protein, 14 aa, Artificial Sequence
  Synthetic Construct
KSLERRKNPLLRKE